(19) United States
(12) Patent Application Publication
(10) Pub. No.: US 2012/0270275 A1
(43) Pub. Date: Oct. 25, 2012

(54) SYSTEMS AND METHODS FOR TREATING BIOMASS AND CALCULATING ETHANOL YIELD

(76) Inventors: Marcus Brian Mayhall FENTON, Cambridgeshire (GB); Tsz Hang Emily HO, Kettering (GB); Robert SCOTT, Godmanchester (GB); Pete THOMPSON, Clinton, CT (US)

(21) Appl. No.: 13/486,865

(22) Filed: Jun. 1, 2012

Related U.S. Application Data
(63) Continuation-in-part of application No. 12/590,129, filed on Nov. 2, 2009.
Application No. 12/590,129 is a continuation-in-part of application No. PCT/GB2008/050210, filed on Mar. 21, 2008.
Application No. PCT/GB2008/050210 is a continuation-in-part of application No. 12/290,700, filed on Oct. 30, 2008, now Pat. No. 8,193,395.
Application No. 12/290,700 is a continuation-in-part of application No. PCT/GB2008/050210, filed on Mar. 21, 2008.
Application No. PCT/GB2008/050210 is a continuation-in-part of application No. PCT/GB2008/050319, filed on May 2, 2008.
Application No. PCT/GB2008/050319 is a continuation-in-part of application No. 12/451,268, filed on May 14, 2010.
Application No. 12/451,268 is a continuation-in-part of application No. 11/658,265, filed on Jan. 24, 2007.

(30) Foreign Application Priority Data
Jul. 29, 2004 (GB) 0416914.0
Jul. 29, 2004 (GB) 0416915.7
Aug. 12, 2004 (GB) 0417961.0
Dec. 24, 2004 (GB) 0428343.8
May 2, 2007 (GB) 0708482.5
Jun. 5, 2007 (GB) 0710659.4

(51) Int. Cl. C12P 7/14; C12M 1/40; G06F 19/00; C12P 19/14

(52) U.S. Cl. 435/99; 435/162; 435/303.3; 435/294.1; 702/25

(57) ABSTRACT

The present invention provides processes, inter alia, for the treatment of a starch-based feedstock. The processes include mixing together a starch-based feedstock and a working fluid to form a slurry, hydrating the starch-based feedstock with the working fluid, adding an enzyme to the slurry, pumping the slurry into a substantially constant diameter passage of a fluid mover, and injecting a high velocity transport fluid into the slurry through one or more nozzles communicating with the passage, thereby further hydrating and heating the starch-based feedstock and dispersing the starch content of the slurry. Apparatuses for carrying out such processes are also provided. Processes for converting starch in feedstocks into oligosaccharides and systems for producing sugars and ethanol using the processes and apparatuses of the invention are also provided. Processes for calculating ethanol yield using the apparatuses are also provided.

### CROSS-REFERENCE TO RELATED APPLICATIONS

This application is a continuation-in-part of and claims benefit to U.S. application Ser. No. 12/590,129 filed on Nov. 2, 2009, which U.S. application is a continuation-in-part of and claims benefit to international application no. PCT/GB2008/050210 filed Mar. 21, 2008, which international application claims benefit to Great Britain application nos. 0708482.5 filed on May 2, 2007, and 0710659.4 filed on Jun. 5, 2007. The present application also claims benefit, as a continuation-in-part, to U.S. application Ser. No. 12/290,700, which was filed on Oct. 30, 2008 (now allowed), which U.S. application claims benefit to international application nos. PCT/GB2008/050210 filed on Mar. 21, 2008 and PCT/GB2008/050319 filed on May 2, 2008, which both international applications claim priority to Great Britain application nos. 0708482.5 filed on May 2, 2007 and 0710659.4 filed Jun. 5, 2007. The present application also claims benefit, as a continuation-in-part, to U.S. application Ser. No. 12/451,268, which was filed on May 14, 2010, which U.S. application is the U.S. national stage of international application no. PCT/GB2008/050319 filed on May 2, 2008. U.S. application Ser. No. 12/451,268 also claims benefit, as a continuation-in-part, to U.S. application Ser. No. 11/658,265, which is identified in more detail below. The present application also claims benefit, as a continuation-in-part, to U.S. application no. 11/658,265 filed Jan. 24, 2007, which U.S. application is the U.S. national stage of international application no. PCT/GB2005/02999 filed Jul. 29, 2005, which international application claims benefit to Great Britain application nos. 0416914.0, which was filed on Jul. 29, 2004, 0416915.7, which was filed on Jul. 29, 2004, 0417961.0, which was filed on Aug. 12, 2004, and 0428343.8, which was filed on Dec. 24, 2004. All of the foregoing applications are incorporated by reference in their entireties as if recited in full herein.

### FIELD OF THE INVENTION

The present invention relates, inter alia, to a biomass treatment process suitable for use in manufacturing alcohol, such as, for example, ethanol for biofuel production, as well as other products such as sugars, sugar syrups or products that are fed into alternative fermentation/reaction routes to make end products other than alcohol. More specifically, the present invention relates to an improved process and apparatus for converting starch-based biomass into sugars. Subsequently, the sugars may undergo a series of processes (such as saccharification, fermentation and distillation) whose end products are, e.g., an alcohol.

### BACKGROUND OF THE INVENTION

The process of converting starch-based biomass into sugars in biofuel production is a multi-step process involving hydration, activation (gelatinisation) and liquefaction (conversion). Hydration means the absorption of water via diffusion into the starch granule. Starch activation is the swelling of starch granules by the absorption of additional water in the presence of heat such that the hydrogen bonds between the starch polymers within the granule loosen and break allowing the polymeric structure to unfold in space in the presence of water. This is an irreversible breakdown of the crystalline structure of the starch, eventually the starch granule ruptures and the starch polymers are dispersed in solution forming a viscous colloidal state. The liquefaction process is the conversion of gelatinised starch into shorter chain polysaccharides (dextrins). Subsequently, the dextrins may undergo saccharification (hydrolysis to small sugar units), fermentation and distillation into alcohol such as ethanol, for example.

Processes used in industry for the conversion of starch-based biomass into sugars typically involve an initial hydration step of mixing ground starch-based feedstock with water to form a slurry. The water may be pre-heated prior to being mixed with the feedstock. The slurry may additionally be heated in a vessel in order to activate the starch, and is then heated again and mixed with a liquefaction enzyme in order to convert the starch to shorter chain sugars.

At present, there are two main processes used in industry for the conversion of starch-based biomass to sugars. In the first process, the activation stage typically uses steam jacketed tanks or steam sparge heating to heat the slurry to the desired temperature typically above 70° C., preferably above 85° C., and hold it at that temperature for 30 to 45 minutes in order to hydrate and gelatinise the starch. A liquefaction enzyme may also be added at this stage to reduce the viscosity of the slurry. At the same time agitation mixers, slurry recirculation loops, or a combination of the two mix the slurry. The slurry is then pumped to a second heated vessel for the liquefaction stage where the gelatinised starch is converted to dextrins. One drawback of the above process is that the temperatures reached in the first vessel are not high enough to fully gelatinise the starch, leading to a reduction in yield.

However, despite the presence of the recirculation pumps these heating methods can result in regions being created in the slurry tank or vessel whose temperature is much greater than the remainder of the tank. In such hydration and gelatinisation processes, starch hydrated early in the process can be damaged if it comes into contact with these high temperature regions, resulting in a lower yield. These arrangements also do not provide particularly efficient mixing, as evidenced by the heat damage problem discussed above.

This first type of conventional process normally uses separate vessels for the activation and conversion stages of the process. Transfer of the slurry from the activation (and hydration) vessel to the conversion stage vessel is normally accomplished using centrifugal pumps, which impart a high shear force on the slurry and can cause further damage to the hydrated gelatinised starch as a result.

The conversion (liquefaction) stage may also use steam- or water-jacketed tanks, or tanks heated by sparge heaters, to raise the temperature of the slurry to the appropriate level for the optimum performance of the enzyme.

In the second method, jet cookers are employed to heat the slurry to temperatures between 105° C. and 110° C. once it has left the activation vessel. The hot slurry is then flashed into a low pressure tank and water vapour is removed. The slurry is then cooled and pumped into the conversion stage vessel. Not only can the slurry suffer the same heat damage as in the activation stage, but the high temperature regions also contribute to limiting the dextrin (sugar) yield from the process. The excessive heat of these regions promotes Maillard reactions, where the sugar molecules are destroyed due to interaction with proteins also present in the slurry. The combination of these Maillard losses with the shear losses from the transfer pumps limits the dextrin yield. A reduced yield of dextrins from the liquefaction process obviously reduces the yields of any subsequent processing stages, such as glucose yield from the saccharification stage, and hence alcohol yield from the fermentation stage. Additionally, the high temperatures caused by the jet cooker denature the liquefaction enzyme such that a second dose of enzyme needs to be added to enable the liquefaction process. This increases the cost of the process as does the energy required for the extra heating and cooling stages. Furthermore, existing liquefaction processes require a long residence time for the slurry in the conversion stage to ensure that as much starch is converted to sugars as possible. This can lead to a longer production process with increased costs.

Thus, there is a need for improved systems and methods for treating and converting starch-based biomass into sugars that may subsequently be converted into, e.g., ethanol, for biofuel production. Moreover, there is a need for improved systems and methods for measuring yield (such as ethanol yield) in the production of biofuels. Traditional methods of measuring yield—which may be defined in the fuel ethanol industry as the volume units of ethanol obtained from a mass unit of grain—rely on averages of the total amount of grain received per month and the total volume of ethanol sold per month. One drawback of this method is that it is inaccurate as it relies on measuring bulk masses of corn and bulk volumes of ethanol. These measurements are difficult to ascertain with precision and are not sensitive enough to provide the spot yield given that they rely on average amounts taken over a lengthy period of time. Checking precise inventory on a more regular basis to predict yield is not practical as parts of each supply of feedstock or ethanol can be rejected or delayed in delivery. Regularly checking precise inventory is also not practical given that it is likely to be time consuming and require a dedicated operator. Another drawback of the above method for measuring yield is that the method prevents a plant operator from responding in a fast manner, either by altering the balance of ingredients or the operating conditions in the plant, given that such yield measurements are only available once a month.

### SUMMARY OF THE INVENTION

Accordingly, one aim of the present invention is to mitigate or obviate one or more of the foregoing disadvantages.

Thus, a first embodiment of the present invention is a process for the treatment of a starch-based feedstock. This process comprises mixing together a starch-based feedstock and a working fluid to form a slurry, hydrating the starch-based feedstock with the working fluid, adding an enzyme to the slurry, pumping the slurry into a substantially constant diameter passage of a fluid mover, and injecting a high velocity transport fluid into the slurry through a nozzle communicating with the passage, thereby heating and further hydrating the starch-based feedstock, and activating the starch content of the slurry.

According to a second embodiment of the present invention, there is provided an apparatus for treating a starch-based feedstock. The apparatus comprises a hydrator/mixer for mixing and hydrating the feedstock with a working fluid to form a slurry and a fluid mover in fluid communication with the first hydrator/mixer. In this embodiment, the fluid mover comprises a passage of substantially constant diameter having an inlet in fluid communication with the first hydrator/mixer and an outlet; and a transport fluid nozzle communicating with the passage and adapted to inject high velocity transport fluid into the passage.

According to a third embodiment of the present invention, there is provided a system for producing ethanol comprising an apparatus according to the present invention, which apparatus is integrated into an ethanol production plant.

According to a fourth embodiment of the present invention, there is provided a process for making ethanol comprising saccharifying and fermenting the activated starch content produced by carrying out a system according to the present invention on a starch-based feedstock.

According to a fifth embodiment of the present invention, there is provided a process for converting a starch contained within a starch-based feedstock into shorter chain polysaccharides by a process according to the present invention.

According to other embodiments of the present invention, there is provided processes, apparatuses and systems for the treatment of a starch-based feedstock. According to certain embodiments, a starch-based feedstock and a working fluid are mixed together to form a slurry. The starch-based feedstock is hydrated with the working fluid. Such mixing and hydrating may take place in a hydrator/mixer. The slurry is preferably heated and/or maintained at a temperature in the range of 55° C.-85° C., and is directed to one or more fluid movers, each having a constant diameter passage, whereby a high velocity transport fluid is injected into the slurry through one or more nozzles communicating with the passage. The slurry or a portion thereof (e.g., the working fluid component) is atomised to form a dispersed droplet flow regime downstream of the one or more nozzles. Such processes, apparatuses, and/or systems preferably target the starch that is more difficult to gelatinise (i.e. starch that typically requires heating to a temperature that is higher than 75° C.), increase yield, and can be used to produce ethanol or non-ethanol products. They may be used in conjunction with a jet or hot cook installation. The fluid movers discussed herein may also pump the slurry (in addition to heating it). Alternatively, a separate pump may be used to move the slurry through the system, in which case less or none of the energy of the fluid mover and corresponding reactor would be used for pumping and more—if not all—of the energy may be dedicated to heating, mixing, hydrating the starch, etc.

According to yet other embodiments of the present invention, a process for calculating ethanol yield during the production of biofuels in a plant is provided. Such a process includes the steps of establishing a composition of dry matter and water making up a mass unit of mash entering into a fermenter that is part of an ethanol production system within the plant, and calculating a mass of dry matter and a mass of wet matter making up the mass unit. An amount of wet corn in the mass unit may be calculated by adding the mass of dry matter and the mass of wet matter. An amount of ethanol produced from the mass unit may also be calculated based on ethanol concentration measurements from the fermenter, and the yield may be determined by dividing the calculated amount of ethanol by the calculated amount of wet corn. One or more of these steps may be implemented using a computer as they rely on stoichiometry and measurements of materials going into and leaving, for example, the fermenter. One or more parameters, such as operating conditions and inputs (e.g., ingredient balance), may be adjusted during production based on the resulting calculation to further improve yield. Examples of such parameters that may be adjusted include the temperature of the slurry, its flow rate and/or throughput, transport fluid speed, process time, pH level, the amount or ratio of feedstock/liquid present in the slurry, the amount of enzyme present and particle size.

### BRIEF DESCRIPTION OF THE DRAWINGS

FIG. 1 is a schematic view of a biofuel processing apparatus.

FIG. 2 is a longitudinal section view through a fluid mover suitable for use in the apparatus shown in FIG. 1, FIG. 8, FIG. 10, or FIG. 11.

FIG. 3 shows a graph of the temperature and pressure profile of a slurry as it passes through the device shown in FIG. 2.

FIG. 4 is a schematic view of part of the processing apparatus shown in FIG. 1, FIG. 8, FIG. 10, or FIG. 11, with various configurations of fluid movers included.

FIG. 5 is a schematic view of part of one embodiment of the processing apparatus according to the present invention.

FIG. 6 is a schematic view of part of another embodiment of the processing apparatus according to the present invention with a recirculation loop included.

FIG. 7 is a longitudinal section view through another embodiment of a fluid mover suitable for use in the apparatus shown in FIG. 1, FIG. 8, FIG. 10, or FIG. 11.

FIG. 8 is a schematic view of a biomass processing apparatus targeting starch that gelatinises at higher temperatures as compared to starch targeted using the apparatus of FIG. 1.

FIG. 9 shows an illustrative graph that plots the temperature range over which starch granules from an exemplary feedstock may gelatinise.

FIG. 10 is a schematic view of a biomass processing apparatus that relies on a jet cook installation.

FIG. 11 is a schematic view of a biomass processing apparatus that relies on a hot cook installation.

FIG. 12 is a schematic view of a sub-system for fermenting and distilling ethanol post-liquefaction.

FIG. 13 is a block diagram view of a process for calculating yield.

### DETAILED DESCRIPTION OF THE INVENTION

The present invention relates, inter alia, to improved processes and apparatuses for converting starch-based biomass into sugars. Accordingly, the processes and apparatuses of the present invention are suitable for use in industrial processes as a first step in the production of an alcohol such as ethanol. One such industrial process is the processing of starch-based biomass for biofuel production. Other applications are the production of ethanol for a wide variety of other uses. For example, ethanol is used as a solvent in the manufacture of varnishes and perfumes; as a preservative for biological specimens; in the preparation of essences and flavourings; in many medicines and drugs; and as a disinfectant and in tinctures (e.g. tincture of iodine). Ethanol is also used as a feedstock in the production of other chemicals, for instance in the manufacture of ethanal (i.e. acetaldehyde) and ethanoic acid (i.e. acetic acid). Because the processes and apparatuses of the present invention relate to an improved process for manufacturing sugars from starch-based biomass, they are also suitable for the production of sugar products, examples of which include dextrose, maltose, glucose and glucose syrup (e.g. corn syrup, widely used in processed foods, which is glucose syrup manufactured from maize), as well as other dextrins (e.g. fructose, maltodextrin, and high fructose syrup). Other examples of non-ethanol products that can be produced from the processes and apparatuses of the present invention include sugar alcohols (e.g. maltitol, xylitol, erythritol, sorbitol, mannitol, and hydrogenated starch hydrolysate), and other commercially useful chemicals, many of which are used in foods and pharmaceuticals. Such sugar products will be produced by processes (such as controlled saccharification steps) after the liquefaction step of the present invention.

There are two types of plant designs currently being built in the industry for making alcohol from starch-based biomass, namely “Dry Mill” and “Wet Mill” plants. Corn dry grind is the most common type of ethanol production in the United States. In the dry grind process, the entire corn kernel is first ground into flour and the starch in the flour is converted to ethanol via fermentation. The other products are carbon dioxide (used in the carbonated beverage industry) and an animal feed called dried distillers grain with solubles.

Corn wet milling is a process for separating the corn kernel into starch, protein, germ and fiber in an aqueous medium prior to fermentation. The primary products of wet milling include starch and starch-derived products (e.g. high fructose corn syrup and ethanol), corn oil, and corn gluten. The apparatuses and processes of the present invention, described in further detail below, may be integrated into any conventional bioethanol plant—either Dry Mill or Wet Mill—in order to improve the efficiency and lower the production costs of such a plant.

Accordingly, one embodiment of the present invention is a process for the treatment of a starch-based feedstock. This process comprises mixing together a starch-based feedstock and a working fluid to form a slurry, hydrating the starch-based feedstock with the working fluid, adding an enzyme to the slurry, moving by, e.g., pumping the slurry into a substantially constant diameter passage of a fluid mover, and injecting a high velocity transport fluid into the slurry through one or more nozzles communicating with the passage, thereby further hydrating the starch-based feedstock and activating the starch content of the slurry.

In this embodiment, the step of injecting a high velocity transport fluid into the slurry may include:

applying a shear force to the slurry;

atomising at least a portion of the slurry to create a dispersed droplet flow regime;

forming a low pressure region downstream of the nozzle; and

generating a condensation shock wave within the passage downstream of the nozzle(s) by condensation of the transport fluid or a mixture of transport fluid and working fluid.

The first hydrating step may further include heating the slurry and/or maintaining it at a first predetermined temperature within a first vessel for a first predetermined period of time. The process may further comprise recirculating the slurry through the first vessel.

The process may further comprise the step of transferring the slurry to a second vessel from the fluid mover, and maintaining the temperature of the slurry in the second vessel for a second predetermined period of time.

The step of transferring the slurry to the second vessel may include passing the slurry through a temperature conditioning unit to raise the temperature of the slurry. Alternatively, this step may include passing the slurry through a low pressure flash tank to reduce the temperature of the slurry.

The process may also include the step of agitating the slurry in the first and/or second vessels for the respective first and second periods of time.

The transport fluid may be a hot, compressible gas, such as, e.g., steam, carbon dioxide, nitrogen, or other like gasses. Preferably, the transport fluid is steam. The transport fluid may be injected at a subsonic or supersonic velocity. The working fluid may be water as defined herein.

The step of injecting the transport fluid may comprise injecting the high velocity transport fluid into the slurry through a plurality of nozzles communicating with the passage. The step of injecting the transport fluid into the slurry may occur on a single pass of the slurry through the fluid mover. The step of injecting the transport fluid into the slurry may also include recirculating the slurry through the fluid mover.

The pumping of the slurry may be carried out using a pump, such as a low shear pump.

In the process according to the present invention, the feedstock may be selected from any starch-based plant material suitable for conversion to, e.g., alcohol, such as ethanol. Preferably, the feedstock is dry milled maize, dry milled wheat, or dry milled sorghum. The feedstock could also include starch stocks derived from potato, oats, barley, rye, rice (or dry milled rice) and cassava.

According to another embodiment of the present invention, there is provided an apparatus for treating a starch-based feedstock. The apparatus comprises a hydrator/mixer for mixing and hydrating the feedstock with a working fluid to form a slurry and a fluid mover in fluid communication with the first hydrator/mixer. In this embodiment, the fluid mover comprises a passage of substantially constant diameter having an inlet in fluid communication with the first hydrator/mixer and an outlet; and a transport fluid nozzle communicating with the passage and adapted to inject high velocity transport fluid into the passage.

The hydrator/mixer may comprise a heater to heat the working fluid and/or the slurry. The hydrator/mixer may comprise a first vessel having an outlet in fluid communication with the inlet of the passage. The heater may comprise a heated water jacket surrounding the first vessel. Alternatively, the heater may be remote from the hydrator/mixer.

The apparatus may further comprise a second vessel having an inlet in fluid communication with the outlet of the passage. The second vessel may include an insulator to insulate the contents of the second vessel. The insulator may comprise a heated water jacket surrounding the second vessel. Alternatively, the insulator may comprise a layer of insulating material covering the exterior of the second vessel.

The apparatus may further comprise a residence tube section having an inlet in fluid communication with the outlet of the passage. The residence tube may include an insulator for insulating the contents of the residence tube as it passes through. Such an insulator may be a layer of insulating material covering the exterior of the residence tube section, or the residence tube may have a heated water jacket surrounding it.

The transport fluid nozzle may be annular and circumscribe the passage. The transport fluid nozzle may have an inlet, an outlet and a throat portion intermediate the inlet and the outlet, wherein the throat portion has a cross sectional area which is less than that of the inlet and the outlet. The passage may be of substantially constant diameter.

The apparatus may further comprise a transport fluid supply adapted to supply transport fluid to the transport fluid nozzle.

The apparatus may comprise a plurality of fluid movers in series and/or parallel with one another, wherein the transport fluid supply is adapted to supply transport fluid to the transport fluid nozzle of each device. The apparatus may comprise a plurality of transport fluid supply lines connecting the transport fluid supply with each nozzle, wherein each transport fluid supply line includes a transport fluid conditioner. The transport fluid conditioner may be adapted to vary the supply pressure of the transport fluid to each nozzle.

Alternatively, the apparatus may comprise a dedicated transport fluid supply for each transport fluid nozzle. Each transport fluid supply may include a transport fluid conditioner. Each conditioner may be adapted to vary the supply pressure of the transport fluid to each respective nozzle.

The apparatus may further comprise a temperature conditioning unit located between the fluid mover and the second vessel, the temperature conditioning unit is adapted to increase the temperature of fluid passing from the device to the second vessel. Alternatively, the apparatus may comprise a low pressure flash tank or other device located between the fluid mover and the second vessel, the flash tank or other device is adapted to reduce the temperature of the fluid passing to the second vessel, as needed.

The apparatus may further comprise a recirculation pipe adapted to allow fluid recirculation between the outlet of the fluid mover and the first vessel, e.g., from downstream of the fluid mover to upstream of the fluid mover.

The apparatus may further comprise a pump, or other suitable device for moving the fluid. For example, the pump may or may not be a low shear pump adapted to pump fluid from the hydrator/mixer to the fluid mover.

The apparatus may further comprise first and second agitators located in the first and second vessels, respectively. The first vessel may include a recirculator for recirculating slurry from the outlet to an inlet thereof.

The apparatus may be integrated into an ethanol production plant for producing ethanol from a feed stock, such as, e.g., a plant as disclosed in the Example or described herein.

In another embodiment, the invention is a system for producing alcohol, e.g., ethanol. The system includes an apparatus according to the present invention, which is integrated into an alcohol, e.g., ethanol, production plant.

In this embodiment, the ethanol production plant may be a dry mill or a wet mill plant. The plant may utilize either a dry grind based feedstock or a wet milling based feedstock. Preferably, the plant is a dry mill, which utilizes a dry grind based feedstock.

Another embodiment of the present invention is a process for making ethanol. This process includes carrying out a system according to the present invention and then saccharifying and fermenting the product to produce, an alcohol, e.g., ethanol. In the present invention, any conventional process for carrying out the saccharifying and fermenting steps, preferably commercial scale processes, are contemplated.

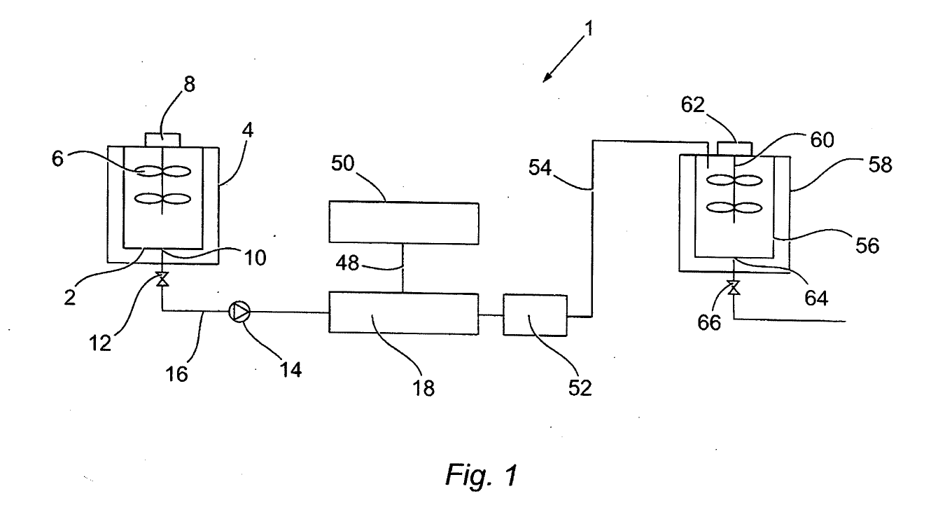

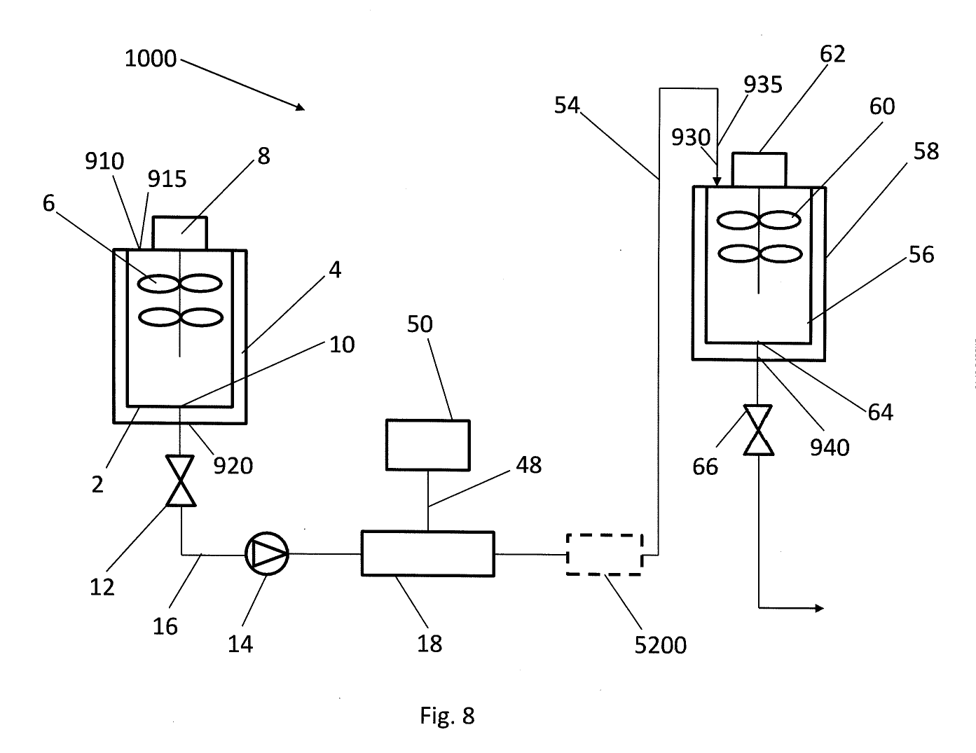

A further embodiment of the present invention is a process for converting a starch contained within a starch-based feedstock into polysaccharides, oligosaccharides and glucose. This process involves carrying out a process according to the present invention, e.g., the process depicted in FIG. 1, FIG. 8 or other similar figures. The addition of, for example, alpha-amylase to make the shorter chain polysaccharides could be paired/followed with the addition of, for example gluco-amylase in order to break the polysaccharides down further to simpler sugars and monosaccharides, such as glucose.

In another embodiment, the invention includes a system and process for calculating and monitoring yield (such as ethanol yield) in the production of biofuels.

The apparatuses and processes of the present invention will now be described in more detail with reference to the figures. Turning now to FIG. 1, it schematically illustrates an apparatus which hydrates and gelatinises the starch from a starch-based feedstock and then makes it more accessible so that it can be converted into shorter chain polysaccharides by, e.g., liquefaction enzymes. The apparatus, generally designated 1, comprises a first vessel 2 acting as a first hydrator/mixer. The first vessel 2 has a heater, which is preferably a heated water jacket 4 which surrounds the vessel 2 and receives heated water from a heated water supply (not shown). In the present invention, the heater may be a traditional heater, a heat exchanger, sparge pipes, hot water injection systems and other like devices/systems well known to those skilled in the art. The vessel 2 also includes an agitator 6 that is powered by a motor 8. The agitator 6 is suspended from the motor 8 so that it lies inside the vessel 2. At the base of the vessel 2 are an outlet 10 and a valve 12 which controls fluid flow from the outlet 10. Downstream of the first vessel 2 is a first supply line 16, the upstream end of which fluidly connects to the outlet 10 and valve 12 whilst the downstream end of the supply line 16 fluidly connects with a reactor 18. A pump 14 may be provided in the supply line 16. The pump 14 may be a centrifugal pump which has been modified in order to reduce shear as fluid is pumped through it.

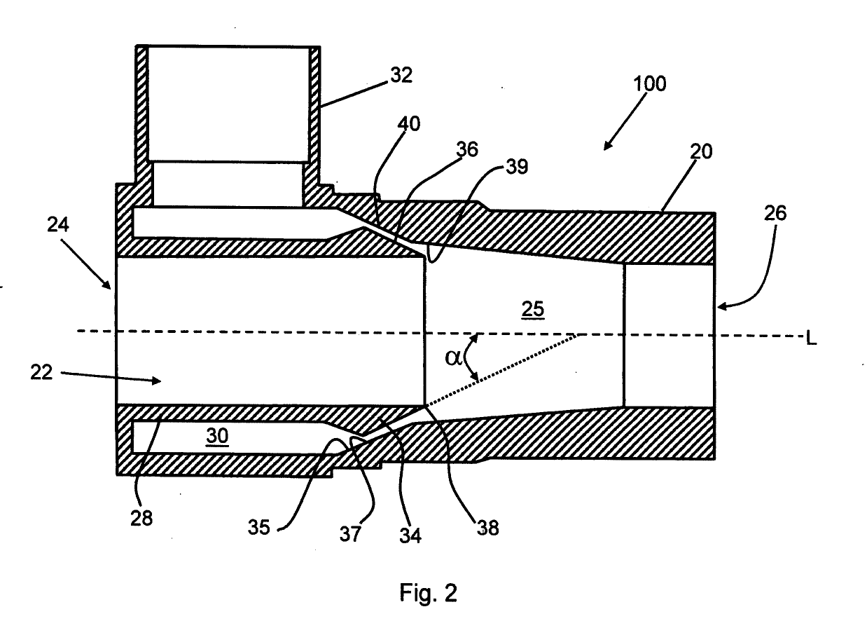

The reactor 18 is formed from one or more fluid movers. A suitable device that may act as a fluid mover is shown in detail in FIG. 2. The fluid mover 100 comprises a housing 20 that defines a passage 22. The passage 22 has an inlet 24 and an outlet 26, and is of substantially constant diameter. The inlet 24 is formed at the front end of a protrusion 28 extending into the housing 20 and defining exteriorly thereof a plenum 30. The plenum 30 has a transport fluid inlet 32. The protrusion 28 defines internally thereof part of the passage 22. The distal end 34 of the protrusion 28 remote from the inlet 24 is tapered on its relatively outer surface at 36 and defines a transport fluid nozzle 38 between it and a correspondingly tapered part 40 of the inner wall of the housing 20. The nozzle 38 is in fluid communication with the plenum 30 and is preferably annular such that it circumscribes the passage 22. The nozzle 38 has a nozzle inlet 35, a nozzle outlet 39 and a throat portion 37 intermediate the nozzle inlet 35 and nozzle outlet 39. The nozzle 38 has convergent-divergent internal geometry as is known in the art, wherein the throat portion 37 has a cross sectional area which is less than the cross sectional area of either the nozzle inlet 35 or the nozzle outlet 39 and where there is a smooth and continuous decrease in cross-sectional area from the nozzle inlet 35 to the throat portion 37 and a smooth and continuous increase in cross-sectional area from the throat portion 37 to the nozzle outlet 39. The nozzle outlet 39 opens into a mixing chamber 25 defined within the passage 22.

Referring once again to FIG. 1, the reactor 18 is connected to a transport fluid supply 50 via a transport fluid supply line 48. The transport fluid inlet 32 for each fluid mover 100 making up the reactor is fluidly connected with the transport fluid supply line 48 for the receipt of transport fluid from the transport fluid supply 50.

Located downstream of the reactor 18 and fluidly connected thereto is a temperature conditioning unit (TCU) 52. The TCU 52 preferably comprises a fluid mover substantially identical to that illustrated in FIG. 2, and will therefore not be described again in detail here. The TCU 52 can either be connected to the transport fluid supply 50 or else it may have its own dedicated transport fluid supply (not shown).

Downstream of the TCU 52 is a second supply line 54, which fluidly connects the outlet of the TCU 52 with a second vessel 56. The second vessel 56 is similar to the first vessel 2, and therefore has a heater, such as, e.g., a heated water jacket 58 which surrounds the vessel 56 and receives heated water from a heated water supply (not shown). The vessel 56 also includes an agitator 60 that is powered by a motor 62. The agitator 60 is suspended from the motor 62 so that it lies inside the vessel 56. At the base of the vessel 56 are an outlet 64 and a valve 66 which controls fluid flow from the outlet 64.

A representative method of processing a starch-based feedstock using the apparatus illustrated in FIGS. 1 and 2 will now be described in detail. Firstly, a ground starch-based feedstock is introduced into the first vessel 2 at a controlled mass addition flow rate. Non-limiting examples of suitable feedstock include dry milled maize, wheat or sorghum. Feedstock may be added by any method, such as manually, automatically, continuously or in batch mode. For instance, in a large production facility the feedstock may be added from a continuous belt feed whilst in a small test rig the feedstock may be added manually. Separately, an enzyme that catalyzes the breakdown of the feedstock is mixed with a working fluid, preferably water, and that working fluid is then added to the feedstock in the vessel 2 to form a slurry and to start to hydrate the feedstock. “Water” in this context is not limited to pure water, but instead is intended to encompass all types of water (e.g. hard or soft water, aqueous solutions etc.) also fluids recovered from a later stage in the processing apparatus, or a combination of the above. An example of a recovered fluid is ‘backset’—a water-based fluid that may contain dissolved solids, solid debris and other soluble or insoluble impurities from the fermenter, which is recovered from the separator after fermentation. Another example is process condensate, which is water recovered from a distillation stage.

As used in the present invention, an “enzyme” or a “liquefaction enzyme”, which are used interchangeably herein, is a naturally occurring or genetically engineered protein that functions as a biochemical catalyst either enabling and/or accelerating a given process, e.g., the breakdown/conversion of the feedstock. The enzymes may be of fungal, bacterial or plant origin. One skilled in the art will recognize that other types of catalysts, such as, e.g., non-natural catalysts, such as metal ions, graphitic carbon, etc., may also be used in the present invention, as well as living organisms such as yeast or bacteria which actively produce enzymes. Preferably, the enzymes of the present invention are typically sourced from the fungus Aspergillis niger or bacteria Bacillus licheniformis. An example of a suitable enzyme is α-amylase, for which a typical level of enzyme activity for the processes of the present invention is between 750 and 824 AGU/g, where enzyme activity is given per unit mass of wet feedstock. The preferred enzyme concentration in the vessel 2 is about 0.09-0.18 ml/kg.

Preferably, the ratio of feedstock to liquid content in the slurry is 20%-40% by weight. Typical α-amylases used in the liquefaction stage have an activity optima when the pH is between about 5.5 and about 6.5. Optionally, one or more pH adjusters and/or surfactants may also be added to the slurry at this point. For instance, process condensate often has a low pH (e.g. 2-3) and once it and the feedstock have been mixed to form a slurry, ammonia may be added to adjust the pH to that required by the enzyme.

Heated water, such as, e.g., recycled hot water recovered from another part of a process plant, is fed into the water jacket 4 surrounding the vessel 2 and the heated water jacket then heats the slurry to a temperature of typically 30° C.-60° C., preferably 45° C.-55° C., and holds the slurry at this temperature for 30-120 minutes so as to hydrate the crystalline regions of the starch granules. The motor 8 drives the agitator 6, which stirs the slurry in the vessel 2 with gentle (i.e. low shear) agitation whilst the slurry is held in the vessel 2. Alternatively, the working fluid may be heated prior to being mixed with the feedstock and the heater 4 in the vessel 2 may then maintain the slurry at the desired temperature. The enzyme may be added into the vessel 2 separately from the working fluid. The enzyme may be added before the slurry has reached the desired temperature.

The slurry is held at the desired temperature in the vessel 2 for a sufficient period of time to allow the starch content to be prepared for full, or substantially full, hydration and gelatinisation. “Sufficient” in this context means the time required for the crystalline, un-gelatinised starch grains in the slurry to absorb as much water as possible. The water being absorbed into the crystallised starch grains acts as a plasticiser, destabilising the hydrogen bonds that help to order the crystal structure. When the slurry has been steeped in the vessel 2 for sufficient time, the valve 12 is opened to allow the slurry to leave the vessel via the outlet 10. As used herein, “steeping,” “steeped,” and other like terms refer to the process of soaking the starch-based biomass as a slurry at a time and temperature in order to facilitate hydration of the un-gelatinised starch therein. The pump 14 pumps the slurry under low shear conditions from the vessel 2 through the first supply line 16 to the reactor 18.

Referring again to FIG. 2, when the slurry reaches the or each fluid mover 100 forming the reactor 18, slurry will pass into the fluid mover 100 through the inlet 24 and out of the outlet 26. Transport fluid, which in this non-limiting example is preferably steam, is fed from the transport fluid supply 50 (FIG. 1) at a preferred pressure of between 5-9 bar to the, or each, transport fluid inlet 32 via transport fluid supply line 48 (FIG. 1). Introduction of the transport fluid through the inlet 32 and plenum 30 causes a jet of steam to issue forth through the nozzle 38 at a very high subsonic or, more preferably, supersonic velocity.

The nozzle outlet 39 opens into a mixing chamber 25 defined within the passage 22. The angle at which the transport fluid exits the transport fluid nozzle 38 affects the degree of shear between it and the feedstock passing through the passage 22, the turbulence levels in the vapour-droplet flow regime and the further development of the fluid flow. The angle α most readily defines the angle of inclination of the transport nozzle 38 to the passage 22. This angle is that formed between the leading edge of the divergent portion of the transport nozzle 38 which is the relatively outer surface 36 of the distal end 34 of the protrusion 28 and the longitudinal axis L of the passage 22. The angle α is preferably between 0° and 70°, more preferably between 0° and 30°.

As the steam is injected into the slurry, a momentum and mass transfer occurs between the two which preferably results in the atomisation of at least part of the slurry to form a dispersed droplet flow regime. This transfer is enhanced through turbulence. “Atomised” in this context should be understood to mean break down into very small particles or droplets. The steam preferably applies a shearing force to the slurry which not only atomises the working fluid component but also helps disrupt the ultrastructure (e.g., cellular structure) of the feedstock suspended in the slurry, such that some or all of the starch granules present are separated from the feedstock and dispersed into the slurry. Free surface area is critical in processing starch granules. For example, based on some simple finite element modelling based on rates of water diffusion and heat conduction into a generic polymer model, when free surface area is reduced from 100% to 70%, the time required for homogenous heating of the granules from 20° C. to 75° C. will be doubled. Similarly, the time required for achieving 80% of the saturated water absorption will at least be doubled when free surface area is reduced to 70%. Thus, atomising the working fluid component of the slurry will greatly speed the rate and completeness of the gelatinisation process.

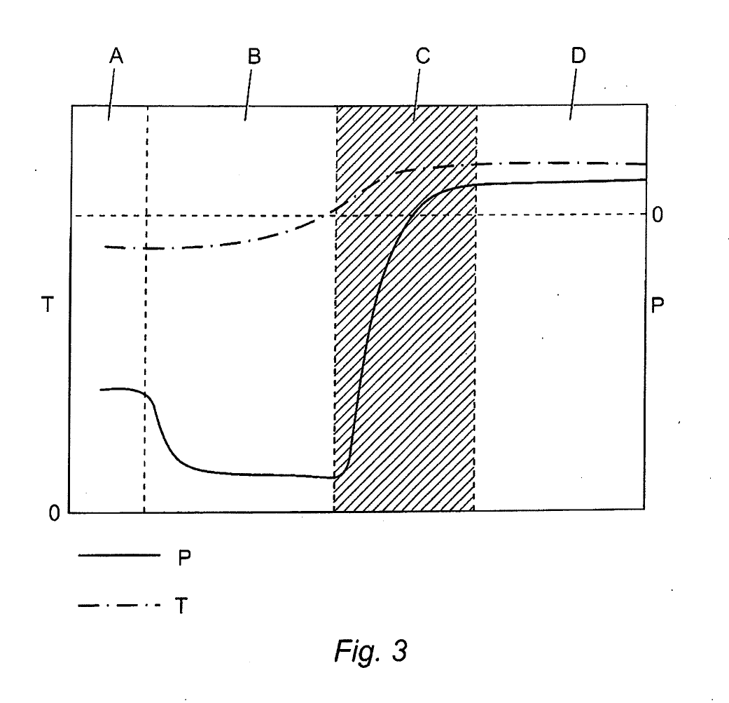

The effects of the process on the temperature and pressure of the slurry can be seen in the graph of FIG. 3, which shows the profile of the temperature and pressure as the slurry passes through various points in the fluid mover 100 of FIG. 2. The graph in FIG. 3 has been divided into four sections A-D, which correspond to various sections of the fluid mover 100. Section A corresponds to the section of the passage 22 between the inlet 24 and the nozzle 38. Section B corresponds to the upstream section of the mixing chamber 25 extending between the nozzle 38 and an intermediate portion of the chamber 25. Section C corresponds to a downstream section of the mixing chamber 25 extending between the aforementioned intermediate portion of the chamber 25 and the outlet 26, while section D illustrates the temperature and pressure of the slurry as it passes through the outlet 26.

The steam is injected into the slurry at the beginning of section B of the FIG. 3 graph. The speed of the steam, which is preferably injected at a supersonic velocity, and its expansion upon exiting the nozzle 38 may cause an immediate pressure reduction. At a point determined by the steam and geometric conditions, and the rate of heat and mass transfer, the steam may begin to condense, further reducing or continuing to maintain the low pressure and causing an increase in temperature. The steam condensation may continue and form a condensation shock wave in the downstream section of the mixing chamber 25. The forming of a condensation shock wave causes a rapid increase in pressure, as can be seen in section C of FIG. 3. Section C also shows that the temperature of the slurry also continues to rise through the condensation of the steam.

As explained above, as the steam is injected into the slurry through nozzle 38 a pressure reduction may occur in the upstream section of the mixing chamber 25. This reduction in pressure forms an at least low pressure zone and possibly a partial vacuum in this upstream section of the chamber 25 adjacent the nozzle outlet 39. Tests have revealed that sub-system pressure (whether in substantial vacuum or not) can be achieved in the chamber 25 as the steam is injected and subsequently condenses. This low pressure region may enhance the starch gelatinisation process.

As previously disclosed herein, the shear force applied to the slurry and the subsequent turbulent flow created by the injected steam disrupts the ultrastructure (e.g., cellular structure) of the feedstock suspended in the slurry, releasing the starch granules from the feedstock. As the slurry passes through the low pressure zone or partial vacuum and condensation shock wave formed in the chamber 25, it is further disrupted by the changes in pressure occurring, as illustrated by the pressure profile in sections B and C of FIG. 3.

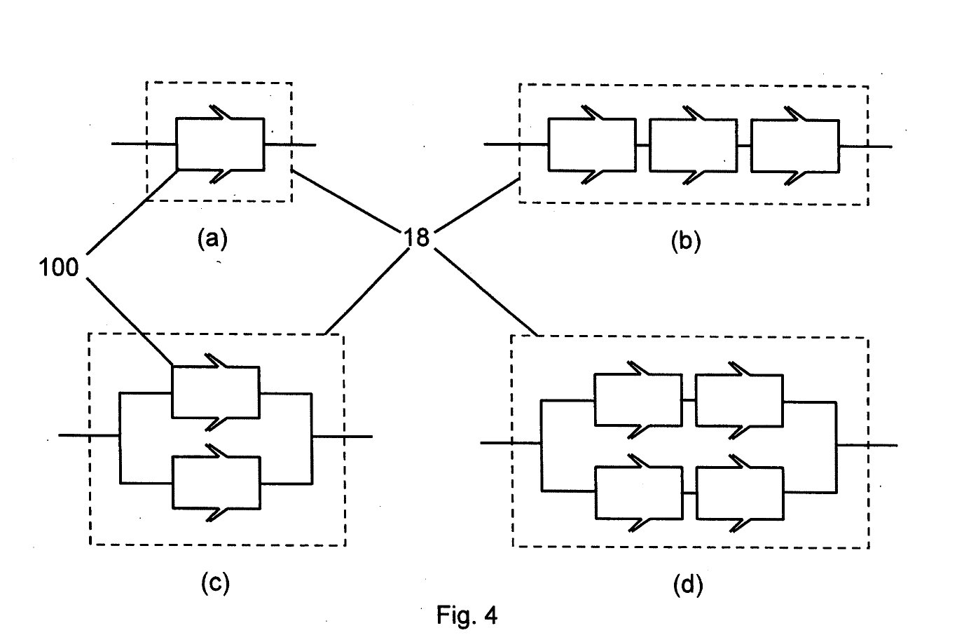

As the starch granules in the feedstock pass into the reactor 18 (FIG. 1), they are almost instantaneously heated and further hydrated resulting in gelatinisation due to the introduction of the steam. The fluid mover(s) 100 making up the reactor 18 simultaneously pump and heat the slurry and complete the hydration and activate or gelatinise the starch content as the slurry passes through. In addition, the reactor 18 mixes the enzyme(s) with the slurry, providing a homogenous distribution and high level of contact with the starch, which is now in a liquid phase. The temperature of the slurry as it leaves the reactor 18 is preferably between 80° C.-86° C. Where the reactor 18 comprises a number of fluid movers in series (e.g., FIG. 4(b)), the pressure of the steam supplied to each fluid mover can be individually controlled by a transport fluid conditioner (not shown) so that the optimum temperature of the slurry for the activity and stability of the liquefaction enzymes is only reached as it exits the last fluid mover in the series. The transport fluid conditioner may be attached directly to the transport fluid supply 50, or else may be located in the transport fluid supply lines 48.

The temperature at which the slurry leaves the reactor 18 is selected to avoid any heat damage to the slurry contents during the activation stage. However, this temperature may be below the temperature for optimal performance of the liquefaction enzyme, and so the temperature of the slurry may need to be raised without subjecting the slurry to excessively high temperatures or additional shear forces. This gentle heating is achieved using the optional TCU 52 downstream of the reactor 18.

As described above, the TCU 52 comprises one or more fluid movers of the type illustrated in FIG. 2. Where there is more than one fluid mover in the TCU 52, they are preferably arranged in series. The pressure of the steam supplied to the fluid mover(s) of the TCU 52 is controlled so that it is comparatively low when compared to that of the steam supplied to the fluid mover(s) 100 of the reactor 18. A preferred steam input pressure for the fluid mover(s) of the TCU is between about 0.5-2.0 bar. Consequently, the transport fluid velocity is much lower so no shear force or condensation shock is applied to the slurry by the injected steam as the slurry passes through the TCU 52. Instead, the TCU 52 merely uses the low pressure steam to gently raise the temperature of the slurry.

Once it has passed through the TCU 52, the slurry is preferably at a temperature of between 83° C.-86° C. The slurry then flows downstream through the second supply line 54 into the second vessel 56. The water jacket 58 of the second vessel receives heated water, which maintains the slurry at the aforementioned temperature. The slurry is held in the second vessel 56 for a sufficient residence time to allow the enzyme to convert or hydrolyse the starch content into oligosaccharides (e.g. maltodextrins). During that residence time, the motor 62 drives the agitator 60 to gently agitate the slurry. It has been found that approximately 30 minutes is a sufficient residence time in the present process, compared with a typical residence time of 120 minutes in existing liquefaction processes. The process of the present invention may also be used to reduce the amount of enzyme required whilst maintaining the slurry in the second vessel 56 for a residence time akin to existing liquefaction processes. The progress of the conversion is monitored during the residence time by measuring the dextrose equivalent (DE) of the slurry. As used herein, “DE” indicates the degree of hydrolysis of starch into shorter chain polysaccharides. Calculating the DE is a simple method of estimating the efficiency of the liquefaction process. The higher the DE, the shorter the average length of the chains and the more efficient the liquefaction process. Typically, the DE value is in the range 1-10 prior to liquefaction and 6-22 after liquefaction. The required DE value depends on the application, those processes that do not require a subsequent fermentation step (such as commercial processes to manufacture sugars) can tolerate much higher DE values. For those processes that do involve a subsequent fermentation step, the required DE value depends substantially on the yeast that the process will use.

At the end of the residence time, the mash (after the liquefaction stage, the slurry is often referred to as a ‘mash’) may be transferred to a fermentation tank (not shown) via the outlet 64 and control valve 66 of the second vessel 56. pH adjustors may also be added at this point via a feed port (not shown) because the glucoamylases and yeasts used in the fermentation stage typically operate at a pH optima of 3.5-4.5. As an example, the pH may be adjusted using phosphoric acid, and/or materials such as urea which also act as nutrient sources for the yeast in the saccharification/fermentation step can be added. Additionally, the mash may be cooled by a cooling device (not shown), such as a heat exchanger, prior to entry into the fermentation tank, because the fermentation stage typically requires much lower temperatures (e.g. 25° C.-35° C.) than the liquefaction stage. Furthermore, a mash diluent (e.g. water or backset) may be added to thin the mash to maintain a consistent density.

Using a fluid mover of the type described herein allows the present invention to heat and mix the starch content of the slurry with the enzyme while avoiding the creation of regions of extreme heat, which can damage the starch content. Prevention of these regions also reduces or eliminates Maillard effects caused by the reaction of proteins with the extracted starch. These reactions can prevent conversion of the starch to sugar and therefore reduce yields. Furthermore, the gentle agitation, mixing, and low shear pumping at a lower temperature also ensures that there are no high shear forces which may damage the enzyme or starch content of the slurry whilst held in a vessel or being transported between vessels. Such damage limits the ultimate glucose yield available from the feedstock.

The fluid mover(s) of the reactor also ensure that the slurry components are more thoroughly mixed than is possible using simple agitator paddles and/or recirculation loops alone. The atomisation of the liquid component of the slurry further ensures a more homogenous mixing of the constituent parts of the slurry than previously possible. This improved mixing increases the efficiency of the enzyme in converting starch to shorter dextrins, reducing the time to achieve the desired DE values in the slurry when compared with existing processes. Another benefit of the processes of the present invention is that, in a continuous flow processing plant with a fixed liquefaction time, the amount of enzyme required to give the desired DE can be reduced. In addition, using the processes of the present invention, higher DE values than possible with existing processes may be achieved.

The shear action and condensation/pressure shock applied to the feedstock component of the slurry when in the reactor further improves the performance of the present invention as this exposes more of the cellular structure of the feedstock. This allows virtually all the starch granules in the feedstock to become accessible, thereby providing improved starch hydrolysis rates compared to conventional processes as the enzymatic reaction is supplemented by the mechanical mixing in the reactor. This also allows the process to provide an accessible starch to sugar conversion ratio of substantially 100% (i.e., close to 100%). The processes of the present invention, therefore, may only require the slurry to pass once through the reactor before it is ready to pass to the second vessel for the conversion stage. Hence, yields are much improved as there is no loss during the process.

Exposing more starch also means that less of the enzyme is needed to achieve the desired DE value of 6-22 before the slurry is transferred to the saccharification and fermentation processes. In addition, the high degree of dispersal of the material in combination with high temperature kills bacteria, thereby reducing losses in any subsequent fermentation process.

It has also been discovered that the processes and apparatuses of the present invention may also improve fermentation rates in the subsequent fermentation process. The improved hydration of the present invention also hydrates some proteins in the feedstock. These hydrated proteins act as additional feedstock to the fermenting yeast, thereby improving the fermenting performance of the yeast.

In summary, the processes and apparatuses of the present invention have been found to provide a number of advantages over existing arrangements. These advantages include an increase of up to 14% in starch to sugar yields, a reduction of up to 50% of the amount of liquefaction enzyme required, a reduction of up to 75% in the residence time for the conversion to take place, and a reduction of up to 30% in the time taken for the subsequent fermentation of the converted sugars into alcohol.

As described above, the reactor 18 may comprise a plurality of fluid movers 100 arranged in series and/or parallel as shown in FIG. 4. Where the reactor comprises groups of four or more devices in series, the slurry need not be maintained in the desired 30° C.-60° C. temperature range whilst being developed in the first vessel. Instead, as each of the devices in the reactor injects high pressure transport fluid into the slurry, the temperature of the slurry as it leaves the first vessel may need only be 20° C.-30° C. in this instance. An antibiotic additive may be added at the same time as the enzyme, into the first vessel 2, and/or after the liquefaction process and prior to the fermentation stage (where present), if desired. For example, an additive port (not shown) could be included in the pipework after the vessel 56 (FIG. 2). Examples of suitable additives are virginiamycin-based and penicillin-based antibiotics. For many such antibiotics, a cooling device (not shown) would need to be incorporated into the pipework downstream of the vessel 56 and prior to the antibiotic additive port in order to cool the mash.

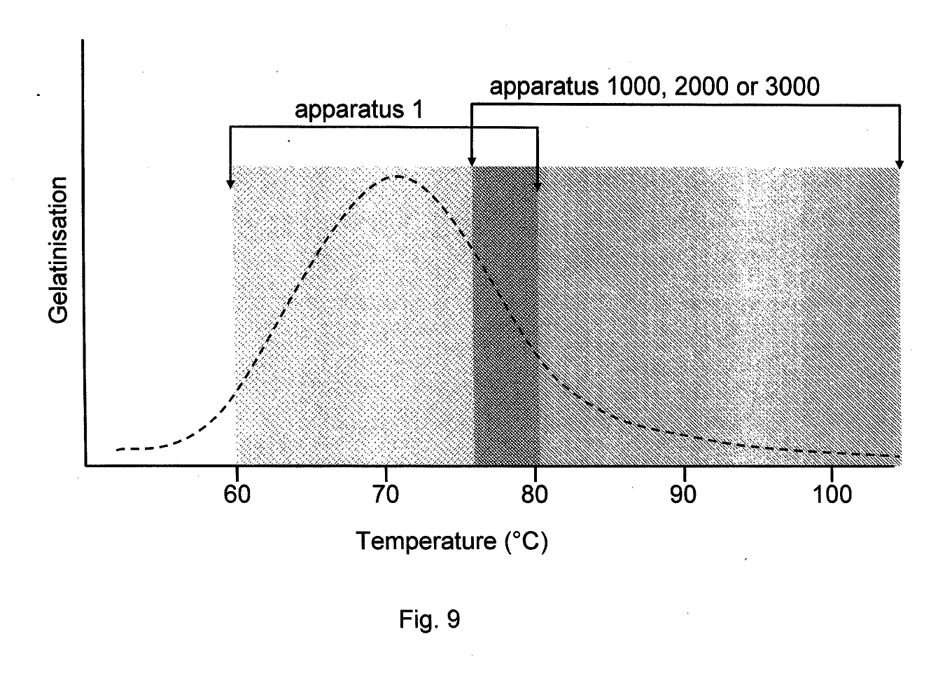

FIG. 8 illustrates an alternative apparatus 1000 for processing biomass in accordance with another embodiment which targets the starch that is more difficult to gelatinise (i.e. starch that typically requires heating to a temperature that is higher than 75° C.). In contrast, apparatus 1 of FIG. 1 and the corresponding process is preferably aimed at gelatinising the majority of starch (i.e. starch that typically requires heating to a temperature in the range of 60° C.-80° C.). For illustration purposes, FIG. 9 shows a schematic graph that plots an exemplary temperature range over which the starch within an exemplary feedstock may gelatinise and illustrates the difference between the starch targeted using apparatus 1 illustrated in FIG. 1 as opposed to the starch targeted using apparatus 1000 illustrated in FIG. 8. Different feedstocks may have starch that gelatinize at different temperatures/ranges. The gelatinisation temperature range of starch depends upon, among other things, the plant type from which the starch originated, the size of the starch grains, the degree of crystallinity and the proportions of amylase and amylopectin in the starch granule. Some types of unmodified native starches start swelling at 55° C., whereas other types start swelling at 85° C. With respect to certain types of starch, using apparatus 1000 of FIG. 8, which preferably targets the type of starch that is more difficult to gelatinise, results in the production of additional ethanol, thereby increasing yield.

Much like the apparatus illustrated in FIG. 1, the alternative apparatus illustrated in FIG. 8 also hydrates and gelatinises the starch from a starch-based feedstock and then makes it more accessible so that it can be converted into shorter chain polysaccharides by, e.g., liquefaction enzymes. However, apparatus 1000 of FIG. 8 targets starch that gelatinises at temperatures in the range of 75° C.-95° C. and/or higher, such as the proportion of starch crystals in ground corn that are hard to gelatinise at lower temperatures. To do so, apparatus 1000 utilizes many of the same components described in FIG. 1. Thus, like numbers have been used for like parts. The main difference is that element 5200 need not be a temperature conditioning unit which raises the slurry's temperature given that the temperature of the slurry leaving reactor 18 is preferably in the range of 80° C.-100° C. Alternatively, element 5200 may be a low pressure flash tank which reduces the slurry temperature from 100° C. to 85° C. More details pertaining to the apparatus illustrated in FIG. 8 and corresponding process will now be described.

Apparatus 1000 of FIG. 8 comprises a first vessel 2 acting as a first hydrator/mixer. The first vessel 2 has a heater, which is preferably a heated water jacket 4 which surrounds the vessel 2 and receives heated water from a heated water supply (not shown). In the present invention, the heater may be a traditional heater, a heat exchanger, sparge pipes, hot water injection systems and other like devices/systems well known to those skilled in the art. The vessel 2 also includes an agitator 6 that is powered by a motor 8. The agitator 6 is suspended from the motor 8 so that it lies inside the vessel 2. At the base of the vessel 2 are an outlet 10 and a valve 12 which controls fluid flow from the outlet 10. Downstream of the first vessel 2 is a first supply line 16, the upstream end of which fluidly connects to the outlet 10 and valve 12 whilst the downstream end of the supply line 16 fluidly connects with a reactor 18. A pump 14 is provided in the supply line 16. Pump 14 may be a centrifugal pump which has been modified in order to reduce shear as fluid is pumped through it. Pump 14 may or may not consist of a low shear pump. As before, reactor 18 may be formed from one or more fluid movers, such as the one shown and described in connection with FIG. 2, which may be arranged in series, or in parallel according to any of the configurations shown in FIG. 4.

Referring once again to FIG. 8, the reactor 18 is connected to a transport fluid supply 50 via a transport fluid supply line 48. The transport fluid inlet 32 for each fluid mover 100 making up the reactor is fluidly connected with the transport fluid supply line 48 for the receipt of transport fluid from the transport fluid supply 50.

Located downstream of the reactor 18 and fluidly connected thereto is a unit 5200. Unit 5200 may be a temperature conditioning unit (TCU), such as one comprising a fluid mover substantially identical to that illustrated in FIG. 2, and may either be connected to the transport fluid supply 50 or else it may have its own dedicated transport fluid supply (not shown). Alternatively, unit 5200 may be a low pressure flash tank as explained above and further below. Yet in another alternative embodiment, no unit need be in place along second supply line 54 which fluidly connects the outlet of reactor 18 with the second vessel 56.

As before, the second vessel 56 is similar to the first vessel 2, and therefore has a heater, such as, e.g., a heated water jacket 58 which surrounds the vessel 56 and receives heated water from a heated water supply (not shown). The vessel 56 also includes an agitator 60 that is powered by a motor 62. The agitator 60 is suspended from the motor 62 so that it lies inside the vessel 56. At the base of the vessel 56 are an outlet 64 and a valve 66 which controls fluid flow from the outlet 64.

A representative method of processing a starch-based feedstock using the apparatus illustrated in FIGS. 8 and 2 will now be described in detail. Firstly, a ground starch-based feedstock is introduced into the first vessel 2 at a controlled mass addition flow rate. Non-limiting examples of suitable feedstock include dry milled maize, wheat or sorghum. The feedstock could also include starch stocks derived from potato, oats, barley, rye, rice (or dry milled rice) and cassava. Feedstock may be added by any method, such as manually, automatically, continuously or in batch mode. For instance, in a large production facility the feedstock may be added from a continuous belt feed whilst in a small test rig the feedstock may be added manually. Separately, an enzyme that catalyzes the breakdown of the feedstock is mixed with a working fluid, preferably water (e.g. hard or soft water, aqueous solutions, etc., fluids recovered from a later stage in the processing apparatus—e.g. backset or water condensate—or a combination of the above), and that working fluid is then added to the feedstock in the vessel 2 to form a slurry and to start to hydrate the feedstock.

Preferably, the ratio of feedstock to liquid content in the slurry is 20%-40% by dry weight. Typical α-amylases used in the liquefaction stage have an activity optima when the pH is between about 5.5 and about 6.5. Optionally, one or more pH adjusters and/or surfactants may also be added to the slurry at this point. For instance, process condensate often has a low pH (e.g. 2-3) and once it and the feedstock have been mixed to form a slurry, ammonia or some other appropriate base may be added to adjust the pH to that required by the enzyme.

Heated water, such as, e.g., recycled hot water recovered from another part of a process plant, is fed into the water jacket 4 surrounding the vessel 2 and the heated water jacket then heats the slurry to a temperature of typically 55° C.-85° C., preferably 65° C.-85° C., and holds the slurry at this temperature for 30-120 minutes so as to hydrate the crystalline regions of the starch granules. The motor 8 drives the agitator 6, which stirs the slurry in the vessel 2 with gentle (i.e. low shear) agitation whilst the slurry is held in the vessel 2. Alternatively, the working fluid may be heated prior to being mixed with the feedstock and the heater 4 in the vessel 2 may then maintain the slurry at the desired temperature. The enzyme may be added into the vessel 2 separately from the working fluid. The enzyme may be added before the slurry has reached the desired temperature.

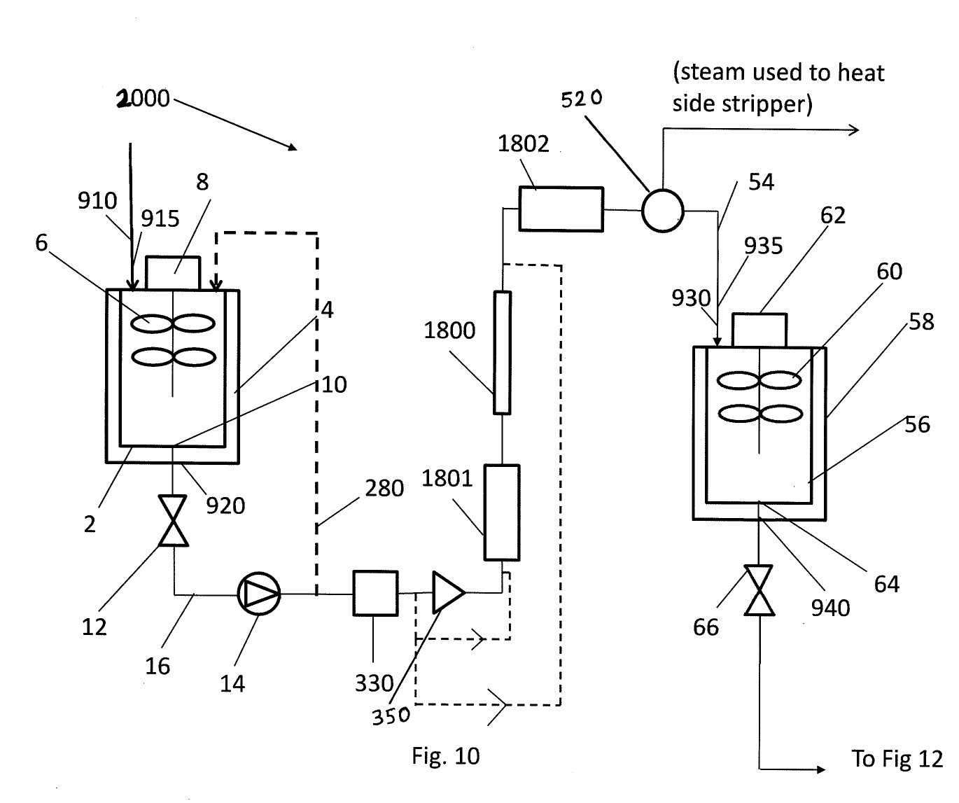

The extent to which the slurry is heated in this embodiment differs from that discussed in connection with FIG. 1. This is because, as discussed before, the embodiments illustrated in FIGS. 8 and 10 are meant to target starch that gelatinises at higher temperatures as shown in FIG. 9, which explains why the slurry is held at a higher temperature in this step (i.e. when the slurry is held in vessel 2), as compared to the temperature at which the slurry is held at the same stage of the process discussed in FIG. 1. Again, the slurry is held at the desired temperature in the vessel 2 for a sufficient period of time to allow the starch content to be prepared for full, or substantially full, hydration and gelatinisation. When the slurry has been steeped in the vessel 2 for sufficient time, the valve 12 is opened to allow the slurry to leave the vessel via the outlet 10. The slurry is then directed from vessel 2 through the first supply line 16 to the reactor 18.

When the slurry reaches reactor 18, slurry will pass into the fluid mover consistent with the description of FIG. 2 above. Again, transport fluid, which in this non-limiting example is preferably steam, is fed from the transport fluid supply 50 at a preferred pressure of between 5-9 bar gauge via transport fluid supply line 48, which causes a jet of steam at a very high subsonic or, more preferably, supersonic velocity. As before, as the steam is injected into the slurry, a momentum and mass transfer—enhanced through turbulence—occurs between the two which preferably results in the atomisation of at least part of the slurry to form a dispersed droplet flow regime. The steam preferably applies a shearing force to the slurry which not only atomises the working fluid component but also disrupts some or all of the ultrastructure (e.g., cellular structure) of the feedstock suspended in the slurry, such that some or all of the starch granules present are separated from the feedstock and dispersed into the slurry. Increasing the free surface area reduces the time required for homogenous heating of the granules, as well as the time required for achieving 80% of the saturated water absorption. Thus, atomising the working fluid component of the slurry and the starch granules will greatly speed the rate and completeness of the gelatinisation process. As before, the effects of the process on the temperature and pressure of the slurry can be seen in the graph of FIG. 3.

As previously disclosed herein, the shear force applied to the slurry and the subsequent turbulent flow created by the injected steam disrupts some or all of the ultrastructure (e.g., cellular structure) of the feedstock suspended in the slurry, releasing the starch granules from the feedstock. As the slurry passes through the low pressure zone (which is at least lower than system pressure and may or not be a partial vacuum) and condensation shock wave formed in the chamber(s) of reactor 18, it is further disrupted by the changes in pressure occurring. As the starch granules in the feedstock pass into the reactor 18, they are almost instantaneously further hydrated and heated, resulting in gelatinisation due to the introduction of the steam. The fluid mover(s) making up the reactor 18 simultaneously pump and heat the slurry and complete the hydration and gelatinisation of the starch content as the slurry passes through. In addition, the reactor 18 mixes the enzyme(s) with the slurry, providing a homogenous distribution and high level of contact with the starch, which is now in a liquid phase. The temperature of the slurry as it leaves the reactor 18 is preferably between 80° C.-100° C. in the embodiment described herein in connection with FIG. 8.

As before, where the reactor 18 comprises a number of fluid movers in series (e.g., FIG. 4(b)), the pressure of the steam supplied to each fluid mover can be individually controlled by a transport fluid conditioner (not shown) so that the optimum temperature of the slurry for the activity and stability of the liquefaction enzymes is only reached as it exits the last fluid mover in the series. The transport fluid conditioner may be attached directly to the transport fluid supply 50, or else may be located in the transport fluid supply lines 48.

In this embodiment, heat damage to the slurry contents below 85° C. in the reactor are of no concern and the temperature of the slurry need not be raised given that the slurry exiting reactor 18 is preferably at or higher than the temperature required for optimal performance of the liquefaction enzyme, namely around 85° C. This explains why there is no need for a TCU that raises the slurry temperature along supply line 54. Instead, given that the temperature of the slurry exiting reactor 18, which could be closer to 100° C., may be higher than the temperature required for optimal performance of the liquefaction enzyme, a low pressure flash tank 5200 may be used to reduce the slurry temperature to between 83° C.-86° C. (preferably 85° C.).

The slurry then flows downstream through the second supply line 54 into the second vessel 56. As described above in connection with FIG. 1, the water jacket 58 of the second vessel of FIG. 8 receives heated water, which maintains the slurry at the aforementioned temperature. The slurry is held in the second vessel 56 for a sufficient residence time to allow the enzyme to convert or hydrolyse the starch content into shorter chain polysaccharides (e.g. dextrins). During that residence time, the motor 62 drives the agitator 60 to gently agitate the slurry. It has been found that approximately 30 minutes is a sufficient residence time in the present process, compared with a typical residence time of 120 minutes in existing liquefaction processes. Thus, the present invention may be used to reduce the process time. Alternatively, the process of the present invention may be used to reduce the amount of enzyme required whilst maintaining the slurry in the second vessel 56 for a residence time akin to existing liquefaction processes. The progress of the conversion is monitored during the residence time by measuring the DE of the slurry. As before, the higher the DE, the shorter the average length of the chains (e.g., the polysaccharide chains) and the more efficient the liquefaction process. Typically, the DE value is in the range 1-10 prior to liquefaction and 6-22 after liquefaction. The required DE value depends on the application, those processes that do not require a subsequent fermentation step (such as commercial processes to manufacture sugars) can tolerate much higher DE values. For those processes that do involve a subsequent fermentation step, the required DE value depends substantially on the second enzyme used in saccharification and on the yeast used in fermentation.

At the end of the residence time, the resulting mash may be transferred to a fermentation tank (not shown) via the outlet 64 and control valve 66 of the second vessel 56 shown in FIG. 8. As before, pH adjustors may also be added at this point via a feed port (not shown) because the glucoamylases and yeasts used in the fermentation stage typically operate at a pH optima of 3.5-4.5. As an example, the pH may be adjusted using phosphoric acid, and/or materials such as urea which also act as nutrient sources for the yeast in the saccharification/fermentation step can be added. Additionally, the mash may be cooled by a cooling device (not shown), such as a heat exchanger, prior to entry into the fermentation tank, because the fermentation stage typically requires much lower temperatures (e.g. 25° C.-35° C.) than the liquefaction stage. Furthermore, a mash diluent (e.g. water or backset) may be added to thin the mash to maintain a consistent density.

As stated above, using a fluid mover of the type described in connection with FIG. 2 increases yield while reducing the amount of liquefaction enzyme required as well as liquefaction and fermentation times. Reactor 18 of FIG. 8 performs best with slurry temperatures of 55° C. and higher. Reactor 18 also performs well with slurry temperatures below 55° C. As described above, reactor 18 may comprise a plurality of fluid movers 100 (FIG. 2) arranged in series and/or parallel as shown in FIG. 4. Where the reactor comprises groups of four or more devices in series, the slurry need not be maintained in the desired 55° C.-85° C. temperature range whilst being developed in the first vessel. Instead, as each of the devices in the reactor injects high pressure transport fluid into the slurry, the temperature of the slurry as it leaves the first vessel may need only be 20° C.-30° C. in this instance.

As before, an antibiotic additive may be added at the same time as the enzyme, into the first vessel 2, and/or after the liquefaction process and prior to the fermentation stage (where present), if desired. For example, an additive port (not shown) could be included in the pipework after the vessel 56. Examples of suitable additives are virginiamycin-based and penicillin-based antibiotics. For many such antibiotics, a cooling device (not shown) would need to be incorporated into the pipework downstream of the vessel 56 and prior to the antibiotic additive port in order to cool the mash.

### Jet Cook Installation

FIG. 10 illustrates an alternative apparatus 2000 for processing biomass in accordance with yet another embodiment which targets the starch that is more difficult to gelatinise (i.e. starch that typically requires heating to a temperature that is higher than 75° C.) and results in the production of additional ethanol, thereby increasing yield. As before, FIG. 9 shows a graph that plots the temperatures at which different starches gelatinise and illustrates the difference between the starch targeted using apparatus 1 illustrated in FIG. 1 as opposed to the starch targeted using apparatus 2000 illustrated in FIG. 10.

Apparatus 2000 illustrates what may be referred to as a jet cook installation. Much like the apparatus illustrated in FIG. 1, the alternative apparatus illustrated in FIG. 10 also hydrates and gelatinises the starch from a starch-based feedstock and then makes it more accessible so that it can be converted into shorter chain polysaccharides by, e.g., liquefaction enzymes. However, apparatus 2000 of FIG. 10 targets starch that gelatinises at temperatures in the range of 75° C.-95° C. and/or higher. To do so, apparatus 2000 utilizes many of the same components described in FIG. 1. Thus, like numbers have been used for like parts. The main difference is that apparatus 2000 includes a recirculation loop 280, a strainer 330, a jet cooker 350 (hence the name of this particular type of installation), and a flash tank 520 (similar to element 5200 of FIG. 8). It is worth noting that the recirculation loop and strainer may also be included in the apparatus of FIGS. 1 and/or 8. Moreover, the reactor configuration used in apparatus 2000 is preferably in two stages having reactor 1801 located after jet cooker 350 and reactor 1802 located before flash tank 520, whereby one or more residence tube(s) 1800 are located between reactors 1801 and 1802. Alternatively, apparatus 2000 may include only one reactor stage, such as 1801 or reactor 1802. More details pertaining to the apparatus illustrated in FIG. 10 and corresponding process will now be described.

Apparatus 2000 comprises a first vessel 2 acting as a first hydrator/mixer. The first vessel 2 has a heater, which is preferably a heated water jacket 4 which surrounds the vessel 2 and receives heated water from a heated water supply (not shown). In the present invention, the heater may be a traditional heater, a heat exchanger, sparge pipes, hot water injection systems and other like devices/systems well known to those skilled in the art. The vessel 2 also includes an agitator 6 that is powered by a motor 8. The agitator 6 is suspended from the motor 8 so that it lies inside the vessel 2. At the base of the vessel 2 are an outlet 10 and a valve 12 which controls fluid flow from the outlet 10. Downstream of the first vessel 2 is a first supply line 16, the upstream end of which fluidly connects to the outlet 10 and valve 12 whilst the downstream end of the supply line 16 fluidly connects with strainer 330. Pump 14 may be provided in the supply line 16. Pump 14 may be a centrifugal pump which has been modified in order to reduce shear as fluid is pumped through it. Pump 14 may or may not consist of a low shear pump.

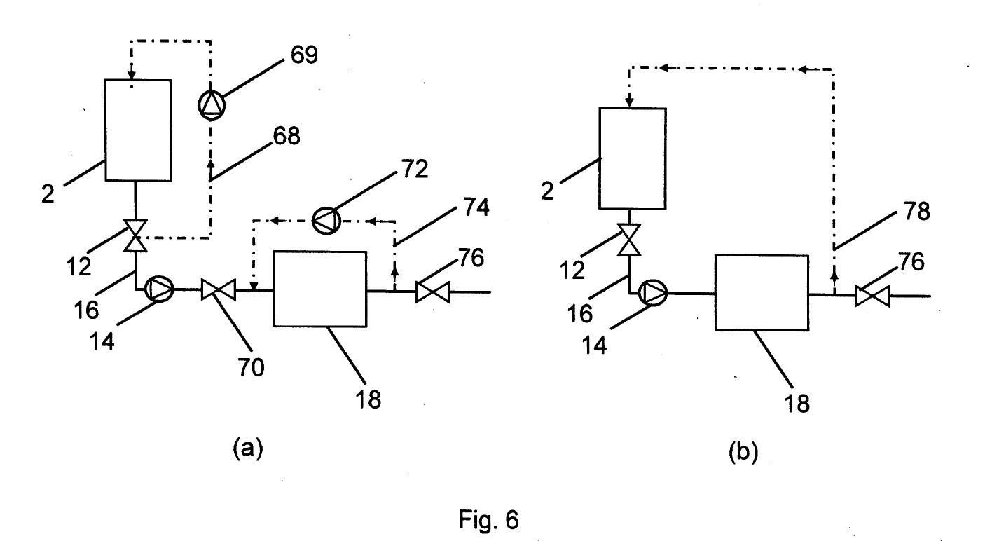

Apparatus 2000 preferably includes recirculation loop 280 which may consist of one or more recirculation pipes that can selectively recirculate slurry through vessel 2 so that slurry can pass through the first vessel more than once, if necessary. Recirculation loop 280 recirculates the slurry through vessel 2 using a pump, which may be pump 14 or another pump that is not shown. Valve 12 prevents the slurry from leaving the vessel until the appropriate conditions have been reached (e.g. slurry temperature). Another valve (not shown) may be located downstream of pump 14 and may function to apportion the slurry such that some passes through the recirculation loop whilst some proceeds into the first supply line 16. Recirculation loop 280 may operate similar to the description below pertaining to the first recirculation loop shown in FIG. 6(a).

The slurry pumped through pump 14 may be passed through strainer 330 to remove large particles and/or other debris (which may be returned to the slurry tank or may be directed to a waste bin for subsequent disposal) and then split into two streams, the first is returned to the slurry tank via recirculation loop 280, the second stream continues to the cooker/reactor(s).

Jet cooker 350 may already be part of the process apparatus into which the processes, systems and/or teachings of the present invention may be retrofit. Jet cooker 350 may be fully open, with or without steam addition. Alternatively a bypass can be built from the exit of strainer 330 to the inlet of reactor 1801 or from the exit of strainer 330 to the inlet of reactor 1802, as shown by the broken lines surrounding these elements in FIG. 10.

As stated above, the reactor configuration used in apparatus 2000 is preferably in two stages, whereby part of the gelatinisation process takes place in reactor 1801 in the first stage, and another part of the gelatinisation process takes place in reactor 1802. In between these stages, the slurry is directed to one or more residence tubes 1800 (preferably 1 tube or 2 tubes in series) where the slurry resides for some time until the appropriate conditions have been reached. For example, the slurry may cool off in residence tube(s) 1800 before being fed into reactor 1802. A residence tube 1800 has a passage, as well as an inlet in fluid communication with the outlet of the passage. The tube may include an insulator for insulating the contents of the residence tube as it passes through. Such an insulator may be a layer of insulating material covering the exterior of the tube section.

Each reactor 1801 and/or 1802 may be formed from one or more fluid movers, such as the one shown and described in connection with FIG. 2, which may be arranged in series, or in parallel according to any of the configurations shown in FIG. 4. As before, each reactor may be connected to a transport fluid supply (not shown) via one or more transport fluid supply line(s) (not shown). Reactors 1801 and 1802 may be connected to the same transport fluid supply or different transport fluid supplies.

Located downstream of reactor 1802 and fluidly connected thereto is flash tank 520, which may be a low pressure flash tank. Downstream of flash tank 520 is a second supply line 54, which fluidly connects the outlet of flash tank 520 with second vessel 56. Moreover, steam resulting from the operation of flash tank 520 (which generally cools the slurry exiting from the two-stage reactor process and therefore results in a heat exchange producing energy that can be used to heat air) may be used to heat a side stripper (not shown) which recovers the trace amounts of ethanol off the bottoms flow. Alternatively, in situations where the temperature of the slurry exiting the reactor need not be decreased (thereby not requiring a flash tank), or where the steam generated from the flash tank is not sufficient in light of the low required decrease in temperature of the slurry, a separate or direct steam source may be used for the side stripper.

As before, the second vessel 56 is similar to the first vessel 2, and therefore has a heater, such as, e.g., a heated water jacket 58 which surrounds the vessel 56 and receives heated water from a heated water supply (not shown). The vessel 56 also includes an agitator 60 that is powered by a motor 62. The agitator 60 is suspended from the motor 62 so that it lies inside the vessel 56. At the base of the vessel 56 are an outlet 64 and a valve 66 which controls fluid flow from the outlet 64.

A representative method of processing a starch-based feedstock using the apparatus illustrated in FIGS. 10 and 2 will now be described in detail. Firstly, a ground starch-based feedstock is introduced into the first vessel 2 at a controlled mass addition flow rate. Non-limiting examples of suitable feedstock include dry milled maize, wheat or sorghum. The feedstock could also include starch stocks derived from potato, oats, barley, rye, rice (or dry milled rice) and cassava. Feedstock may be added by any method, such as manually, automatically, continuously or in batch mode. For instance, in a large production facility the feedstock may be added from a continuous belt feed whilst in a small test rig the feedstock may be added manually. Separately, an enzyme—e.g., α-amylase—that catalyzes the breakdown of the feedstock is mixed with a working fluid, which is preferably recovered from a later stage in the processing apparatus—e.g. backset and/or water condensate—or any other suitable working fluid described above. The working fluid is then added to the feedstock in the vessel 2 to form a slurry and to start to hydrate the feedstock.

Preferably, the ratio of feedstock to liquid content in the slurry is 20%-40% by dry weight. Typical α-amylases used in the liquefaction stage have an activity optima when the pH is between about 5.5 and about 6.5. Optionally, one or more pH adjusters and/or surfactants may also be added to the slurry at this point. For instance, process condensate often has a low pH (e.g. 2-3) and once it and the feedstock have been mixed to form a slurry, ammonia or some other appropriate base may be added to adjust the pH to that required by the enzyme.

Heated water, such as, e.g., recycled hot water recovered from another part of a process plant, is fed into the water jacket 4 surrounding the vessel 2 and the heated water jacket then heats the slurry to a temperature of typically 55° C.-85° C., preferably 65° C.-85° C., and holds the slurry at this temperature for 30-120 minutes so as to hydrate the crystalline regions of the starch granules. Also, recycled hot water may be used in making up the slurry. The slurry may be recirculated through loop 280 into vessel 2 until the target slurry temperature has been reached and/or so as to increase the agitation within the tank whilst increasing the residence time The motor 8 drives the agitator 6, which stirs the slurry in the vessel 2 with gentle (i.e. low shear) agitation whilst the slurry is held in the vessel 2. Alternatively, the working fluid may be heated prior to being mixed with the feedstock and the heater 4 in the vessel 2 may then maintain the slurry at the desired temperature. The enzyme may be added into the vessel 2 separately from the working fluid. The enzyme may be added before the slurry has reached the desired temperature.

The extent to which the slurry is heated in this embodiment differs from that discussed in connection with FIG. 1 and resembles the one discussed in FIG. 8. This is because, as discussed before, the embodiment illustrated in FIG. 8 is meant to target starch that gelatinises at higher temperatures, which explains why the slurry is held at a higher temperature in this step (i.e. when the slurry is held in vessel 2), as compared to the temperature at which the slurry is held at the same stage of the process discussed in FIG. 1. Again, the slurry is held at the desired temperature in the vessel 2 for a sufficient period of time to allow the starch content to be prepared for full, or substantially full, hydration and gelatinisation. When the slurry has been steeped in the vessel 2 for sufficient time, the valve 12 (or another valve controlling the recirculation loop) is opened to allow the slurry to be directed from vessel 2 through the first supply line 16 to the strainer 330, followed by jet cooker 350.

Residence tube 1800 may store and allow residence at the immediate temperature, or to increase the time to allow the full condensation of steam at high temperature. When the slurry reaches reactor 1801, slurry will pass into the fluid mover consistent with the description of FIG. 2 above. Again, transport fluid, which in this non-limiting example is preferably steam, is fed from a transport fluid supply at a preferred pressure of between 5-9 bar gauge via a transport fluid supply line (the transport fluid source and line are not shown in this figure but are similar to elements 50 and 48 of FIGS. 1 and 8), which causes a jet of steam at a very high subsonic or, more preferably, supersonic velocity. As before, as the steam is injected into the slurry, a momentum and mass transfer—enhanced through turbulence—occurs between the two which preferably results in the atomisation of at least part of the slurry to form a dispersed droplet flow regime. The steam preferably applies a shearing force to the slurry which not only atomises the working fluid component but also disrupts some or all of the ultrastructure (e.g., cellular structure) of the feedstock suspended in the slurry, such that some or all of the starch granules present are separated from the feedstock and dispersed into the slurry. Again, increasing the free surface area reduces the time required for homogenous heating of the granules, as well as the time required for achieving 80% of the saturated water absorption. Thus, atomising the working fluid component of the slurry and the starch granules will greatly speed the rate and completeness of the gelatinisation process. As before, the effects of the process on the temperature and pressure of the slurry can be seen in the graph of FIG. 3.

As previously disclosed herein, the shear force applied to the slurry and the subsequent turbulent flow created by the injected steam disrupts some or all of the ultrastructure (e.g., cellular structure) of the feedstock suspended in the slurry, releasing the starch granules from the feedstock. As the slurry passes through the low pressure zone (which is at least lower than system pressure and may or not be a partial vacuum) and condensation shock wave formed in the chamber(s) of reactor 1801, it is further disrupted by the changes in pressure occurring. As the starch granules in the feedstock pass into the reactor 1801, they are almost instantaneously further hydrated and heated, resulting in gelatinisation due to the introduction of the steam. The fluid mover(s) making up the reactor 1801 simultaneously assist in pumping and heat the slurry and complete the hydration and gelatinisation of the starch content as the slurry passes through. In addition, the reactor 1801 mixes the enzyme(s) with the slurry, providing a homogenous distribution and high level of contact with the starch, which is now in a liquid phase. The temperature of the slurry as it enters reactor 1801 is preferably around 85° C., and the temperature of the slurry as it leaves reactor 1801 is preferably between 85° C.-105° C. in the embodiment described herein in connection with FIG. 10. Reactor 1802 preferably operates in a similar fashion as described in connection with reactor 180, except that the temperature of the slurry as it leaves reactor 1802 is preferably higher (e.g., between 90° C.-120° C.).

In this embodiment, heat damage to the slurry contents below 85° C. in the reactor are of no concern and the temperature of the slurry need not be raised given that the slurry exiting reactor 1802 is preferably at or higher than the temperature required for optimal performance of the liquefaction enzyme, namely around 85° C. Thus, flash tank 520 may be used to reduce the slurry temperature to between 83° C.-86° C. (preferably 85° C.).

The slurry then flows downstream through the second supply line 54 into the second vessel 56. As described above in connection with FIGS. 1 and 8, the water jacket 58 of the second vessel of FIG. 10 receives heated water, which maintains the slurry at the aforementioned temperature. The slurry is held in the second vessel 56 for a sufficient residence time to allow for the conversion or hydrolysation of the starch content into shorter chain polysaccharides (e.g. dextrins). As before, α-amylase may be used for this purpose. However, a lower dose may be utilized in this step in vessel 56 as compared to earlier in the process in vessel 2. Alternatively, no enzymes need be utilized in vessel 56. During the residence time, the motor 62 drives the agitator 60 to gently agitate the slurry. It has been found that approximately 30 minutes is a sufficient residence time in the present process, compared with a typical residence time of 120 minutes in existing liquefaction processes. Thus, production process time may be reduced using apparatus 2000. Alternatively, using process 2000, the amount of enzyme required may be reduced whilst maintaining the slurry in the second vessel 56 for a residence time akin to existing liquefaction processes. The progress of the conversion is monitored during the residence time by measuring the DE of the slurry. As before, the higher the DE, the shorter the average length of the chains (e.g., the polysaccharide chains) and the more efficient the liquefaction process. Typically, the DE value is in the range 1-10 prior to liquefaction and 6-22 after liquefaction.

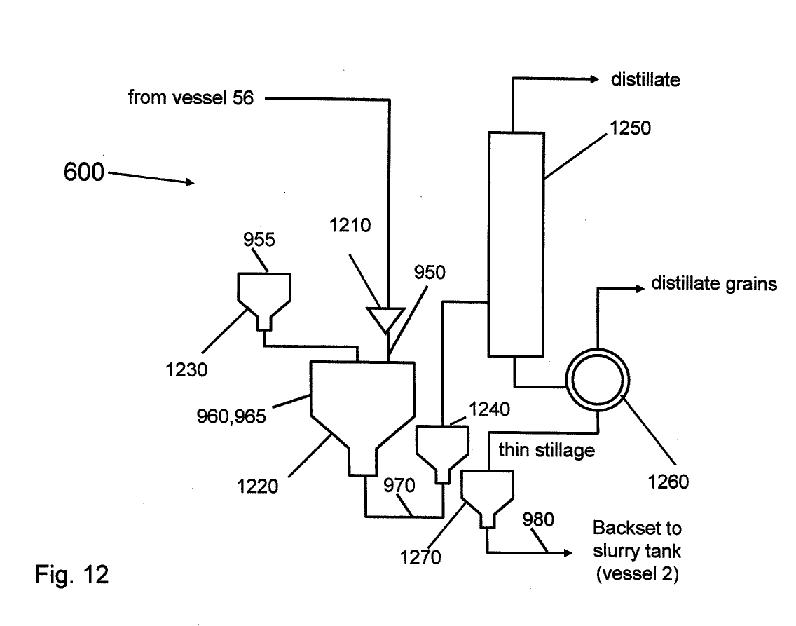

As stated above, using a fluid mover of the type described in connection with FIG. 2 increases yield of sugars from starch, while reducing the amount of liquefaction enzyme required as well as liquefaction and possibly fermentation times. Reactors 1801 and 1802 of FIG. 10 perform best with slurry temperatures of about 85° C. As described above, reactor 1801 and/or reactor 1802 may comprise a plurality of fluid movers 100 (FIG. 2) arranged in series and/or parallel as shown in FIG. 4. As before, an antibiotic additive may be added at the same time as the enzyme, into the first vessel 2, and/or after the liquefaction process and prior to the fermentation stage, if desired. For example, an additive port (not shown) could be included in the pipework after the vessel 56. Examples of suitable additives are virginiamycin-based and penicillin-based antibiotics. For many such antibiotics, a cooling device (not shown) would need to be incorporated into the pipework downstream of the vessel 56 and prior to the antibiotic additive port in order to cool the mash. At the end of the residence time, the resulting mash may be transferred for fermentation and/or distillation, consistent with the post-liquefaction process described in connection with FIG. 12.

### Hot Cook Installation

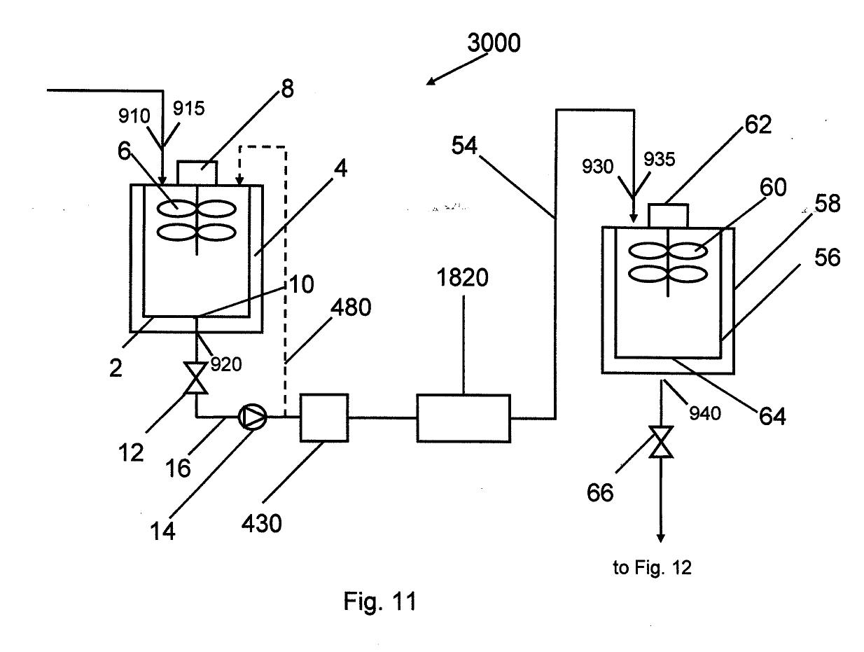

FIG. 11 illustrates an alternative apparatus 3000 for processing biomass in accordance with yet another embodiment which targets the starch that is more difficult to gelatinise (i.e. starch that typically requires heating to a temperature that is higher than 75° C.) and results in the production of additional ethanol, thereby increasing yield. As before, FIG. 9 shows a graph that plots the temperatures at which different starches gelatinise and illustrates the difference between the starch targeted using apparatus 1 illustrated in FIG. 1 as opposed to the starch targeted using apparatus 3000 illustrated in FIG. 11.

Apparatus 3000 illustrates what may be referred to as a hot cook installation. Much like the apparatus illustrated in FIG. 1, the alternative apparatus illustrated in FIG. 11 also hydrates and gelatinises the starch from a starch-based feedstock and then makes it more accessible so that it can be converted into shorter chain polysaccharides by, e.g., liquefaction enzymes. However, apparatus 3000 of FIG. 11 targets starch that gelatinises at temperatures in the range of 75° C.-95° C. and/or higher, such as ground corn, dry milled maize, dry milled wheat, or dry milled sorghum, as well as and starch stocks derived from potato, oats, barley, rye, rice (or dry milled rice) and cassava. To do so, apparatus 3000 utilizes many of the same components described in FIG. 1. Thus, like numbers have been used for like parts. The main difference is that apparatus 3000 includes a recirculation loop 480, a strainer 430, and does not include element 52 (i.e. a TCU). As before, the recirculation loop and strainer may also be included in the apparatus of FIG. 1, FIG. 8 and/or FIG. 10. More details pertaining to the apparatus illustrated in FIG. 11 and corresponding process will now be described.

Apparatus 3000 comprises a first vessel 2 acting as a first hydrator/mixer. The first vessel 2 has a heater, which is preferably a heated water jacket 4 which surrounds the vessel 2 and receives heated water from a heated water supply (not shown). In the present invention, the heater may be a traditional heater, a heat exchanger, sparge pipes, hot water injection systems and other like devices/systems well known to those skilled in the art. The vessel 2 also includes an agitator 6 that is powered by a motor 8. The agitator 6 is suspended from the motor 8 so that it lies inside the vessel 2. At the base of the vessel 2 are an outlet 10 and a valve 12 which controls fluid flow from the outlet 10. Downstream of the first vessel 2 is a first supply line 16, the upstream end of which fluidly connects to the outlet 10 and valve 12 whilst the downstream end of the supply line 16 fluidly connects with strainer 430. Pump 14 may be provided in the supply line 16. Pump 14 may be a centrifugal pump which has been modified in order to reduce shear as fluid is pumped through it. Pump 14 may or may not consist of a low shear pump.

Apparatus 3000 preferably includes recirculation loop 480 which may consist of one or more recirculation pipes that can selectively recirculate slurry through vessel 2 so that slurry can pass through the first vessel more than once, if necessary. Recirculation loop 480 recirculates the slurry through vessel 2 using a pump, which may be pump 14 or another pump that is not shown. Valve 12 prevents the slurry from leaving the vessel until the appropriate conditions have been reached (e.g. slurry temperature). Another valve (not shown) may be located downstream of pump 14 and may function to apportion the slurry such that some passes through the recirculation loop whilst some proceeds into the first supply line 16. Recirculation loop 480 may operate similar to the description below pertaining to the first recirculation loop shown in FIG. 6(a).

The slurry pumped through pump 14 may be passed through strainer 430 to remove large particles and/or other debris (which may be returned to the slurry tank or may be directed to a waste bin for subsequent disposal) and then split into two streams, the first is returned to the slurry tank via recirculation loop 480, the second stream continues to the reactor.

Reactor 1820 used in apparatus 3000 may be formed from one or more fluid movers, such as the one shown and described in connection with FIG. 2, which may be arranged in series, or in parallel according to any of the configurations shown in FIG. 4. As before, the reactor may be connected to a transport fluid supply (not shown) via a transport fluid supply line (not shown). Slurry exiting from reactor 1820 is transported to second vessel 56 through supply line 54.

As before, the second vessel 56 is similar to the first vessel 2, and therefore has a heater, such as, e.g., a heated water jacket 58 which surrounds the vessel 56 and receives heated water from a heated water supply (not shown). The vessel 56 also includes an agitator 60 that is powered by a motor 62. The agitator 60 is suspended from the motor 62 so that it lies inside the vessel 56. At the base of the vessel 56 are an outlet 64 and a valve 66 which controls fluid flow from the outlet 64.

A representative method of processing a starch-based feedstock using the apparatus illustrated in FIGS. 11 and 2 will now be described in detail. Firstly, a ground starch-based feedstock is introduced into the first vessel 2 at a controlled mass addition flow rate. Non-limiting examples of suitable feedstock include dry milled maize, wheat or sorghum. The feedstock could also include starch stocks derived from potato, oats, barley, rye, rice (or dry milled rice) and cassava. Feedstock may be added by any method, such as manually, automatically, continuously or in batch mode. For instance, in a large production facility the feedstock may be added from a continuous belt feed whilst in a small test rig the feedstock may be added manually. Separately, an enzyme—e.g., α-amylase, preferably thermostable up to 95° C. α-amylase—that catalyzes the breakdown of the feedstock is mixed with a working fluid, which is preferably recovered from a later stage in the processing apparatus—e.g. backset and/or water condensate—or any other suitable working fluid described above. The working fluid is then added to the feedstock in the vessel 2 to form a slurry and to start to hydrate the feedstock.

Preferably, the ratio of feedstock to liquid content in the slurry is 20%-40% by dry weight. Typical α-amylases used in the liquefaction stage have an activity optima when the pH is between about 5.5 and about 6.5. Optionally, one or more pH adjusters and/or surfactants may also be added to the slurry at this point. For instance, process condensate often has a low pH (e.g. 2-3) and once it and the feedstock have been mixed to form a slurry, ammonia or some other appropriate base may be added to adjust the pH to that required by the enzyme.

Heated water, such as, e.g., recycled hot water recovered from another part of a process plant, is fed into the water jacket 4 surrounding the vessel 2 and the heated water jacket then heats the slurry to a temperature of typically 55° C.-85° C., preferably 65° C.-85° C., and holds the slurry at this temperature for 30-120 minutes so as to hydrate the crystalline regions of the starch granules. The slurry temperature at entry to the reactor may be 65°-85° C., whereas the exit temperature may be 80° C.-90° C. The slurry may be recirculated through loop 480 into vessel 2 so that slurry until the target slurry temperature has been reached. The motor 8 drives the agitator 6, which stirs the slurry in the vessel 2 with gentle (i.e. low shear) agitation whilst the slurry is held in the vessel 2. Alternatively, the working fluid may be heated prior to being mixed with the feedstock and the heater 4 in the vessel 2 may then maintain the slurry at the desired temperature. The enzyme may be added into the vessel 2 separately from the working fluid. The enzyme may be added before the slurry has reached the desired temperature.

The extent to which the slurry is heated in this embodiment differs from that discussed in connection with FIG. 1 and resembles the one discussed in FIGS. 8 and 10. This is because, as discussed before, the embodiments illustrated in FIGS. 8, 10 and 11 are meant to target starch that gelatinises at higher temperatures as shown in FIG. 9, which explains why the slurry is held at a higher temperature in this step (i.e. when the slurry is held in vessel 2), as compared to the temperature at which the slurry is held at the same stage of the process discussed in FIG. 1. Again, the slurry is held at the desired temperature in the vessel 2 for a sufficient period of time to allow the starch content to be prepared for full, or substantially full, hydration and gelatinisation. When the slurry has been steeped in the vessel 2 for sufficient time, the valve 12 (or another valve controlling the recirculation loop) is opened to allow the slurry to be directed from vessel 2 through the first supply line 16 to the strainer 430.

When the slurry reaches reactor 1820, slurry will pass into the fluid mover consistent with the description of FIG. 2 above. Again, transport fluid, which in this non-limiting example is preferably steam, is fed from a transport fluid supply at a preferred pressure of between 5-9 bar gauge via a transport fluid supply line (the transport fluid source and line are not shown in this figure but are similar to elements 50 and 48 of FIGS. 1 and 8), which causes a jet of steam at a very high subsonic or, more preferably, supersonic velocity. As before, as the steam is injected into the slurry, a momentum and mass transfer—enhanced through turbulence—occurs between the two which preferably results in the atomisation of at least part of the slurry to form a dispersed droplet flow regime. The steam preferably applies a shearing force to the slurry which not only atomises the working fluid component but disrupts some or all of the ultrastructure (e.g., cellular structure) of the feedstock suspended in the slurry, such that some or all of the starch granules present are separated from the feedstock and dispersed into the slurry. Again, Increasing the free surface area reduces the time required for homogenous heating of the granules, as well as the time required for achieving 80% of the saturated water absorption. Thus, atomising the working fluid component of the slurry and the starch granules will greatly speed the rate and completeness of the gelatinisation process. As before, the effects of the process on the temperature and pressure of the slurry can be seen in the graph of FIG. 3.

As previously disclosed herein, the shear force applied to the slurry and the subsequent turbulent flow created by the injected steam disrupts some or all of the ultrastructure (e.g., cellular structure) of a proportion of the feedstock suspended in the slurry, releasing some or all of the starch granules from the feedstock. As the slurry passes through the low pressure zone (which is at least lower than system pressure and may or not be a partial vacuum) and condensation shock wave formed in the chamber(s) of reactor 1820, it is further disrupted by the changes in pressure occurring. As the starch granules in the feedstock pass into the reactor 1820, they are almost instantaneously further hydrated and heated, resulting in gelatinisation due to the introduction of the steam. The fluid mover(s) making up the reactor 1820 simultaneously pump and heat the slurry and complete the hydration and gelatinisation of the starch content as the slurry passes through. In addition, the reactor 1820 mixes the enzyme(s) with the slurry, providing a homogenous distribution and high level of contact with the starch, which is now in a liquid phase. The temperature of the slurry as it enters reactor 1820 is preferably around 65° C.-85° C., and the temperature of the slurry as it leaves reactor 1820 is preferably around 80° C.-90° C. in the embodiment described herein in connection with FIG. 11.

The slurry then flows downstream through the second supply line 54 into the second vessel 56. As described above in connection with FIGS. 1, 8 and 10, the water jacket 58 of the second vessel of FIG. 11 receives heated water, which maintains the slurry at the aforementioned temperature. The slurry is held in the second vessel 56 for a sufficient residence time to allow for the conversion or hydrolysation of the starch content into shorter chain polysaccharides (e.g. dextrins). Preferably, no enzymes need be added in vessel 56. During the residence time, the motor 62 drives the agitator 60 to gently agitate the slurry. It has been found that approximately 30 minutes is a sufficient residence time in the present process, compared with a typical residence time of 120 minutes in existing liquefaction processes. Thus, the production process using apparatus 3000 obviates the need to rely on enzymes whilst maintaining the slurry in the second vessel 56 for a residence time that is shorter than existing liquefaction processes. The progress of the conversion is monitored during the residence time by measuring the DE of the slurry. As before, the higher the DE, the shorter the average length of the chains (e.g. the polysaccharide chains) and the more efficient the liquefaction process. Typically, the DE value is in the range 1-10 prior to liquefaction and 6-22 after liquefaction.

As stated above, using a fluid mover of the type described in connection with FIG. 2 increases yield while reducing the amount of liquefaction enzyme required as well as liquefaction and fermentation times. Reactor 1820 of FIG. 11 performs best with slurry temperatures of about 85° C. As described above, reactor 1820 may comprise a plurality of fluid movers 100 (FIG. 2) arranged in series and/or parallel as shown in FIG. 4. As before, an antibiotic additive may be added at the same time as the enzyme, into the first vessel 2, and/or after the liquefaction process and prior to the fermentation stage, if desired. For example, an additive port (not shown) could be included in the pipework after the vessel 56. Examples of suitable additives are virginiamycin-based and penicillin-based antibiotics. For many such antibiotics, a cooling device (not shown) would need to be incorporated into the pipework downstream of the vessel 56 and prior to the antibiotic additive port in order to cool the mash. At the end of the residence time, the resulting mash may be transferred for fermentation and/or distillation, consistent with the post-liquefaction process described in the section that follows in connection with FIG. 12.

### Post-Liquefaction

After the gelatinised starch has been converted into shorter chain polysaccharides (e.g. dextrins) in vessel 56, the resulting mash undergoes saccharification, fermentation and distillation in order to produce ethanol. A portion of this post-liquefaction process, which includes fermentation and distillation, is described herein in connection with the sub-system 600 illustrated in FIG. 12. It is worth noting that sub-system 600 may be part of—or added to—any apparatus 1, 1000, 2000, and/or 3000 illustrated in FIGS. 1, 8, 10 and 11, respectively. In other words, the output of vessel 56 used in any of the processes depicted in these figures may be fed into sub-apparatus 600 of FIG. 12.

Sub-apparatus 600 includes, inter alia, mash cooler 1210, fermenter 1220, yeast prop tank 1230, beer well 1240, beer column 1250, centrifuge 1260, and thin stillage tank 1270. The mash resulting from the liquefaction in vessel 56 is fed into mash cooler 1210. The mash which is fed from vessel 56 may be introduced via an outlet and control valve (such as outlet 64 and control valve 66 shown in FIG. 1, 8, 10 or 11) disposed on vessel 56. Although not shown, vessel 56 depicted in FIGS. 10 and 11 may include similar control valves. Mash cooler 1210 may be a heat exchanger which cools the mash content because the fermentation stage typically requires much lower temperatures (e.g. 25° C.-35° C.) than the liquefaction stage.

Fermenter 1220, which is a vessel in which saccharification/fermentation of the mash content may take place, is located downstream of mash cooler 1210. Yeast prop tank 1230, which is also coupled to fermenter 1220, supplies yeast (or any other fermenting microorganism(s)) in order for the saccharification/fermentation processes to take place in fermenter 1220. Other additives well known in the fermentation arts may also be provided. For example, a mash diluent (e.g. water or backset) may be added to thin the mash to maintain a consistent density.

After fermentation is complete, the product is dropped into well 1240. Well 1240 is preferably a beer well that feeds into beer column 1250, so as to distil the alcohol, i.e., separate the ethanol and most of the water from the other fermentation products. This produces the ethanol distillate as shown in FIG. 12, as well as stillage which is fed into centrifuge 1260. The stillage is processed by centrifuge 1260 which produces solid distillers grains and thin stillage. The resulting distillers grains can be used as livestock (animal) feed. As for the thin stillage, it is be fed to thin stillage tank 1270 from where it is recycled as backset used in vessel 2 in any of apparatus 1, 1000, 2000 or 3000 as discussed above.

### Ethanol Calculator

Another aspect of the present invention pertains to improved systems and methods for measuring yield (such as ethanol yield) in the production of biofuels using any apparatus or related process described above and in connection with FIGS. 1, 8, and 10-12. These systems and methods result in yield calculations that are more accurate than traditional methods. Moreover, these systems and methods allow plant operators to respond and make adjustments on a relatively quick basis in order to improve yield. Rather than basing yield on the average mass of grain received and the volume of ethanol sold, the systems and methods of the present invention rely on stoichiometric computations based on other parameters, such as lab measurements, that can be ascertained at different stages of the process. As a result, the yield calculations are accurate, quick and can be made often given that they can be calculated using one or more computers in real-time (or near real-time) based on actual process measurements taken repetitively at frequent intervals, or even continuously.

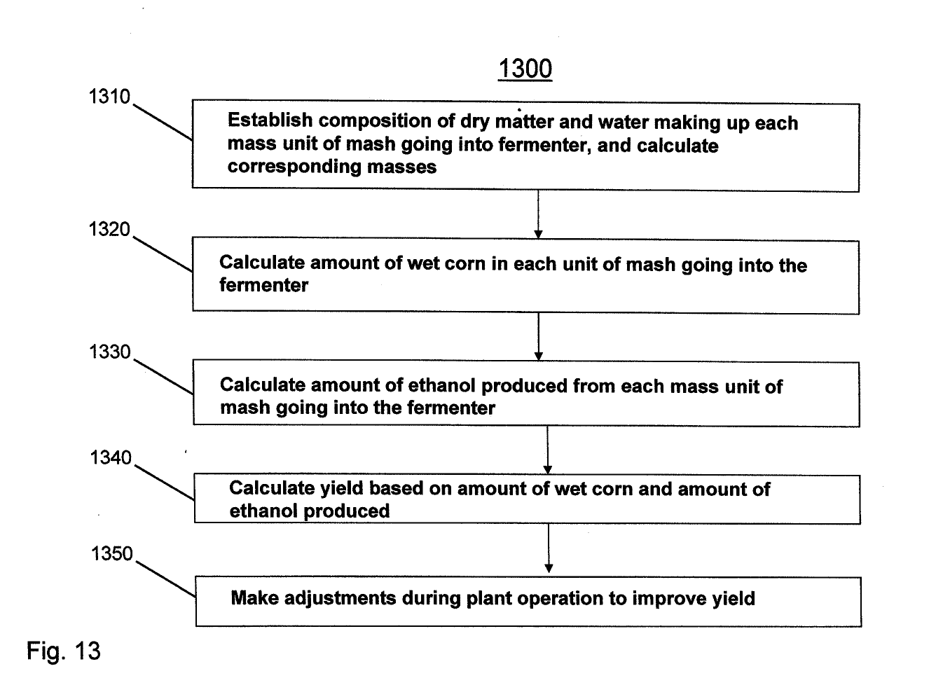

According to a preferred embodiment, the basis for the yield calculation is each unit of mass going into the fermenter (e.g. component 1220 of FIG. 12) before the fermentation process begins. The process 1300 depicted in FIG. 13 for calculating yield can be performed using a computing device (e.g. a general purpose—or specially tailored—computer programmed to perform the steps identified herein), and can be summarized as follows. At step 1310, the composition of dry matter and water making up each mass unit of mash going into the fermenter is established, and the calculation of various masses of dry matter (D) and water (W) may be implemented using equations [1]-[9] below. The mash is hydrolysed prior to entering into the fermenter that is part of the production system. This enables the calculation of the amount of wet corn in each unit of mash going into the fermenter (X_{c}), which is performed at step 1320 based on the equation [11] below. At step 1330, which can be performed concurrently with, after, or before step 1320, the amount of ethanol that can be produced from each mass unit of mash going into the fermenter (X_{EtOH}) can be calculated based on the equations [12]-[16] below. These equations involve stoichiometric determinations and rely on measurements of materials going into and leaving, for example, the fermenter. The measurements relied on preferably include ethanol and dissolved solids concentration, water mass balances and beer density. Some of these measurements (e.g., the beer density ρ_{beer}) may be calculated and others may be measured (e.g., the ethanol concentration C_{EtOH,final}). Finally, yield can be calculated at step 1340 based on the amount of wet corn determined at step 1320 and the amount of ethanol produced at step 1330 based on the equation [17] below.

Process 1300 relies on actual process measurements taken at different stages of the biomass treatment process. In particular, steps 1310 through 1340 and the corresponding equations below rely on various parameters, which are identified in List 1 below and which can be ascertained at different stages in FIGS. 8 and 10-12, e.g., through sensors and/or appropriate measuring devices. More specifically, List 1 identifies each point in FIGS. 8 and 10-12 at which the corresponding parameter that is listed adjacent to that point can be measured. For example, as can be seen below, W_{pc}—i.e. the mass of water in the process condensate (which is part of the composition making up each mass unit of mash going into the fermenter and which is used first for calculating the remainder of the composition) can be measured at point 915. As another example, W_{yp}—i.e. the mass of fresh water added to yeast prop tank 1230—can be measured at point 955.
910 Corn composition=starch & moisture
915

$$\% backset = \frac{b}{b + p}$$

where b=backset, p=process condensate water
920 Temperature of the slurry tank, coming out of the slurry tank
930 Mash mass flow rate
935 Temperature of the mash going into the liquefaction tank
940 Mash solid content
950 Sample point here for “liquefaction sample” for solids content going to fermenter
955 Fresh water added to yeast prop tank
960 Volume/Mass of the total content of the materials filling the fermenter
965 Time for filling each fermenter (there are usually several) (e.g. approx 12 hours). This is used to calculate the total ethanol production from each fermenter after yield has been established. This is also used to calculate the dilution in the fermenter due to the water added in the yeast pot.
970 The ethanol concentration of the fermenter content when it drops (finishes) as measured using an HPLC giving C_{EtOH,final}.
980 Solid content of the backset going into the slurry tank

List 1

The following outlines the composition of each mass unit of mash going into the fermenter:

W_{b}=a₆′W_{pc} [1]

D_{b}=a₄a₆′W_{pc} [2]

Wₒₛa₇′W_{pc} [3]

Dₒₛ=a₅a₇′W_{pc} [4]

D_{c}=a₃W_{c} [5]

### Superseded by:

$${W}_{c} = \frac{( {a}_{1}^{′} {a}_{6}^{′} + {a}_{1}^{′} + {a}_{1}^{′} {a}_{7}^{′} - {a}_{4} {a}_{6}^{′} - {a}_{5} {a}_{7}^{′} ) {W}_{pc}}{[ {a}_{3} - {a}_{1}^{′} + 0.029 {a}_{2} ( {a}_{3} + ) ( 1 + {a}_{1}^{′} ) ]} \qquad [ 6 ]$$

D_{c}+W_{c}+D_{b}+W_{b}+Dₒₛ+Wₒₛ+W_{pc}+Wₛₜₘ+W_{yp}+D_{yp}=1 (kg or lb) [7]

### Where:

D is the mass of dry matter
W is the mass of water
c is from the corn
b is from the backset
os is from another stream
pc is from process condensate
yp is from yeast prop
stm is from steam addition
a₂ is % starch in wet maize
aₙ are constants from dry matter measurements, that is measured by 3 hours loss on drying in an oven of 105-110° C.; and from the ratio of different streams making up the water going into the slurry tank.

List 2

$$\begin{matrix}e . g . {a}_{1} = \frac{dry matter in mash entering fermenter}{total mass in mash entering fermenter} & [ 8 ] \\ and {a}_{1}^{′} = \frac{dry matter in mash entering fermenter}{water in mash entering fermenter - steam added} \\ {a}_{1}^{′} = \frac{{D}_{c} + {W}_{hydrolysis to dextrin} + {D}_{b} + {D}_{os}}{{W}_{c} + {W}_{pc} + {W}_{b} + {W}_{os} - {W}_{hydrolyssis to dextrin}} defines equation [ 6 ] \\ {a}_{1}^{′} = \frac{{a}_{3} {W}_{c} + 0.029 {a}_{2} ( {a}_{3} + 1 ) {W}_{c} + {a}_{4} {a}_{6}^{′} {W}_{pc} + {a}_{5} {a}_{7}^{′} {W}_{pc}}{{W}_{c} + {W}_{pc} + {a}_{6}^{′} {W}_{pc} + {a}_{7}^{′} {W}_{pc} - 0.029 {a}_{2} ( {a}_{3} + 1 ) {W}_{c}} & [ 9 ]\end{matrix}$$

(a₁′a₆′+a₁′+a₁′a₇′−a₄a₆′−a₅a₇′)W_{pc}=[a₃−a₁′+0.029a₂(a₃+1)(1+a₁′)]W_{c}

As shown further below in relation to equation [20],

W_{hydrolysis to dextrin}=0.029a₂(D_{c}+W_{c})=0.029a₂(a₃+1)W_{c} [10]

The amount of steam, Wₛₜₘ, added by the reactor in any of apparatus 1, 1000, 2000, or 3000 (in FIG. 1, 8, 10, or 11) is normally recorded in a plant, or it can be estimated by energy balance (to be explained further below). As a result, the full composition of each mass unit of mash going into a fermenter can be defined; firstly W_{pc}, then W_{b}, D_{b}, Wₒₛ, Dₒₛ, W_{c}, and finally D_{c}.

The amount of wet corn in each mass unit of mash going into the fermenter, X_{c}, is hence known.

X_{c}=D_{c}+W_{c} [11]

The mass of water in the fermenter at the end of fermentation, M_{water,final} can be estimated from the mass of water added into the fermenter, and the mass of water consumed during fermentation (based on stoichiometry to be explained in equations [18] to [20]).

$$\begin{matrix}{M}_{water , final} = {M}_{water into fermenter} - {M}_{water , hydrolysis} \\ = {M}_{water into fermenter} - \frac{0.08}{0.5114 * 1.08} {C}_{EtOH , final} {V}_{beer}\end{matrix} \qquad [ 12 ]$$

The volume of liquid, V_{beer}, at the end of fermentation, is the sum of the volumes of the liquids and the volume of the dissolved solids:

$$\begin{matrix}{V}_{beer} = \frac{{M}_{water , final} + {M}_{EtOH , final}}{{ρ}_{beer}} + \frac{{M}_{ds , final}}{{ρ}_{ds , final}} \\ = \frac{{M}_{water , final} + {C}_{EtOH , final} {V}_{beer}}{{ρ}_{beer}} + \frac{{C}_{ds , final} {V}_{beer}}{{ρ}_{ds , final}}\end{matrix} \qquad [ 13 ]$$

### Where

M stands for mass in kg,

V stands for volume in litre

ρ stands for density in kg/litre

C stands for concentration in kg/litre or g/m litre

### And

EtOH, final stands for ethanol at the end of fermentation

water, final stands for water at the end of fermentation

beer stands for the ethanol and water liquid mixture at the end of fermentation

ds, final stands for dissolved solids at the end of fermentation

Combining equations [12] and [13] results in determining the volume of the liquid in the fermenter at the end of fermentation in terms of the mass of water added into the fermenter initially:

$${V}_{beer} = \frac{{M}_{water into fermenter}}{{ρ}_{beer} - 0.855 {C}_{EtOH , final} - \frac{{ρ}_{beer} {C}_{ds , final}}{{ρ}_{ds , final}}} \\ And because \\ \begin{matrix}\frac{{M}_{water into fermenter}}{{M}_{mash into fermenter}} = & \frac{{M}_{water in mash into fermenter} + {M}_{water into yeast prop}}{{M}_{mash into fermenter}} \\ = & \frac{{M}_{water in mash into fermenter}}{{M}_{mash into fermenter}} + \\ & \frac{{M}_{water in mash into fermenter}}{{M}_{mash into fermenter}} \times \\ & \frac{{M}_{water into yeast prop}}{{M}_{water in mash into fermenter}} \\ = & \frac{{M}_{water in mash into fermenter}}{{M}_{mash into fermenter}} \\ & ( 1 + \frac{{M}_{water into yeast prop}}{{M}_{water in mash into fermenter}} ) \\ = & ( 1 - {a}_{1} ) ( 1 + \frac{{M}_{water into yeast prop}}{{M}_{water in mash into fermenter}} )\end{matrix} \qquad [ 14 ]$$

From equation [14]:

$$\begin{matrix}\frac{{V}_{beer}}{{M}_{mash into fermenter}} = \frac{\frac{{M}_{water into fermenter}}{{M}_{mash into fermenter}}}{{ρ}_{beer} - 0.855 {C}_{EtOH , final} - \frac{{ρ}_{beer} {C}_{ds , final}}{{ρ}_{ds , final}}} \\ = \frac{( 1 - {a}_{1} ) ( 1 + \frac{{M}_{water into yeast prop}}{{M}_{water in mash into fermenter}} )}{{ρ}_{beer} - 0.855 {C}_{EtOH , final} - \frac{{ρ}_{beer} {C}_{ds , final}}{{ρ}_{ds , final}}}\end{matrix} \qquad [ 15 ]$$

As an example, if the density of dissolved solids is:

$$\begin{matrix}{ρ}_{ds} = 1.2 g \text{/} ml ( assume dissolved solids are similar to protein ) \\ = \frac{( 1.2 g \text{/} ml ) ( 1000 ml \text{/} l ) ( 2.2 lbs \text{/} kg ) ( 3.785 l \text{/} gal )}{1000 g \text{/} kg} \\ = 9.992 lb \text{/} gal\end{matrix}$$

$$\begin{matrix}{C}_{ds} = 3 \% = 0.030 g \text{/} ml \\ = \frac{( 0.03 g \text{/} ml ) ( 1000 ml \text{/} l ) ( 2.2 lbs \text{/} kg ) ( 3.785 g \text{/} gal )}{1000 g \text{/} kg} \\ = 0.250 lb \text{/} gal\end{matrix}$$

From [15], it is possible to express the amount of ethanol produced from each unit mass of mash going into the fermenter, X_{EtOH}, as:

$$\begin{matrix}{X}_{EtOH} = \frac{{M}_{EtOH , final}}{{M}_{mash into fermenter}} \\ = \frac{{C}_{EtOH , final} {V}_{beer}}{{M}_{mash into fermenter}} \\ = \frac{{C}_{EtOH , final} ( 1 - {a}_{1} ) ( 1 + \frac{{M}_{water into yeast prop}}{{M}_{water in mash into fermenter}} )}{{ρ}_{beer} - 0.855 {C}_{EtOH , final} - \frac{{ρ}_{beer} {C}_{ds , finak}}{{ρ}_{ds , final}}}\end{matrix} \qquad [ 16 ]$$

Finally, from equations [11] and [16], yield can be calculated:

$$yield = \frac{{X}_{EtOH}}{{X}_{c}} \qquad [ 17 ]$$

The stoichiometry relied on above is based on the following:

(i) starch hydrolysis chemistry,

$${( {C}_{6} {H}_{10} {O}_{5} )}_{n} + n ( {H}_{2} O ) + m ( {H}_{2} O ) \to n ( {C}_{6} {H}_{12} {O}_{6} )$$

$$1 kg Starch 0.11 kg water 0.00 kg water 1.11 kg glucose$$

$$and$$

$${C}_{24} {H}_{42} {O}_{21} + 3 ( {H}_{2} O ) + m ( {H}_{2} O ) \to n ( {C}_{6} {H}_{12} {O}_{6} )$$

$$1 kg {DP}_{4} 0.08 kg water 0.00 kg water 1.08 kg glucose$$

$$\begin{matrix}{M}_{glucose} = \frac{180}{162} {M}_{starch} = 1.11 {M}_{starch} & [ 18 ] \\ {M}_{glucose} = 1.08 {M}_{dextrin} & [ 19 ] \\ {M}_{dextrin} = 1.029 {M}_{glucose} & [ 20 ]\end{matrix}$$

Where m, the branching ratio for the particular carbohydrate, is ˜ 1/100 for corn starch, and where each of M_{glucose}, M_{starch} and M_{dextrin} is the mass of glucose, starch and dextrin respectively;
(ii) glucose fermentation to ethanol by yeast

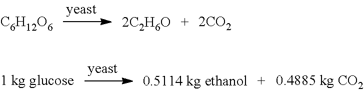

An estimation of steam addition, Wₛₜₘ, if required, can be performed based on an energy balance.

Wₛₜₘ (kg)×Enthalpy of steam (kJ/kg)=Mass of wet corn (kg)×Enthalpy of starch gelatinisation (kJ/kg corn)+Mass of slurry (kg)×temperature rise (deg C)×Heat capacity of the slurry (kJ/kg slurry/deg C)

As shown above, the steam addition Wₛₜₘ depends on the initial and final (e.g. 85° C.) temperatures of the slurry, and the thermal properties of the slurry, which in turn can be derived from the % corn in the slurry.

The mass of wet corn needed to calculate steam addition is approximated as follows:

$$\begin{matrix}\begin{matrix}approximate mass \\ of wet corn\end{matrix} = & ( \frac{1 + {a}_{3}}{{a}_{3}} ) * \\ & approximate mass of dry corn \\ = & \frac{1 + {a}_{3}}{{a}_{3}} * \\ & ( \% dry matter in mash - \\ & \% water in mash * \\ & \% backset * \\ & \% solid in backset ) \\ = & \frac{( 1 + {a}_{3} )}{{a}_{3}} [ {a}_{1} ( 1 - {a}_{1} ) * \\ & \% backset * \\ & \% solid in backset ]\end{matrix} \\ \qquad [ 21 ]$$

Once the steam addition is estimated, it is used to calculate the exact composition of the mash going into the fermenter (including corn) in equations [7] and [9].

Although the specific embodiment and examples discussed in the foregoing are described in the context of measuring ethanol yield from a particular kind of starch, a process similar to that described in FIG. 13 may be used to calculate other yield based on any processed biomass (e.g., maize, wheat or sorghum), even though some of the constants, parameters and inputs may differ, the general methodology for measuring yield may be the same. Moreover, although the embodiment discussed herein is described in the context of measuring ethanol yield, a process similar to that described in FIG. 13 (e.g. steps 1310 through 1340) may be used to calculate other the yield for other fuels or products resulting from the processing of biomass, including processes that use any apparatus 1, 1000, 2000, or 3000.

In addition, process 1300 may include additional optional steps which allow plant operators to respond and make adjustments during plant operation to improve yield based on the calculation produced in step 1340. For example, at step 1350, operating conditions and/or materials inputs may be adjusted. Examples of operating conditions that may be altered include the temperature exit of the slurry from the reactor, the mash process flow rate and/or throughput, the transport fluid speed, the process time and the pH level. Examples of materials/ingredient inputs that may be altered include the amount or ratio of feedstock/liquid present in the slurry, the solids content or the working fluid content of the slurry/mash, the amount of enzyme present and particle size.

### Non-Ethanol & Non-Fuel Production

Although the embodiments discussed above are described in the context of ethanol production, the present invention is not so limited, and in fact, can be used to produce a variety of polysaccharides and sugars from starch-based biomass. For example, as discussed above, the processes and apparatuses of the present invention are also suitable for the production of a wide variety of commercially useful chemicals derived from starch. Products that are made from starch include: dextrins (e.g. fructose, maltodextrin, glucose syrups, corn syrups), dextrose, maltose, and sugar alcohols (e.g. maltitol, xylitol, erythritol, sorbitol, mannitol, hydrogenated starch hydrolysate). Many of these products, which are shorter chain sugars, are used in foods and pharmaceuticals.

A simple example of a starch-derived sugar/oligosaccharide mixture is corn syrup, which is made from maize, and is widely used in food products as a natural sweetener. Corn syrup is used in, for example, cookies, crackers, sauces, cereals, flavoured yogurts, ice cream, preserved meats, canned fruits and vegetables, soups, beers, soft drinks, and many others. Sugar alcohols are popular for use as sweeteners, particularly since they aren't usually absorbed in the bloodstream, so they are widely used in diet foods and foods for diabetics. They are also used to mask the taste of some high-intensity sweeteners.

The apparatus 1000 depicted in FIG. 8 may be used to produce such non-ethanol products from starch. The process described above in connection with FIG. 8 can be largely relied on for such production, although the enzymatic breakdown of starch to sugars in the liquefaction step (i.e. in vessel 56) may involve the use of one or more enzymes, such as α-amylase to break the starch into shorter chains of sugars, and then glucoamylase to break it down to even simpler sugars such as glucose.

Creation of some of these non-ethanol compounds may require further processing after the liquefaction step. For example, some starch-derived sugars and/or polysaccharides may be further chemically, enzymatically, and/or biologically treated to create other commercially useful compounds. For instance, glucose can be converted to a variety of compounds: cyclic and acyclic polyols, aldehydes, ketones, acids, esters, and ethers which can then be used industrially. Polyols such as sorbitol, on the other hand, can be made by fermentation processes similar to that used to make ethanol. Sorbitol is widely used to make surfactants and emulsifiers, which are used in a wide variety of applications, including food products.

Starch-derived products are not just used in the food industry, they may be used to manufacture synthetic polymers including plastics, ingredients in detergents, etc. Of significant interest to many manufacturers is that such starch-derived compounds can be biodegradeable.

Glucose may also be derived from the liquefaction and saccharification of starch to act as the carbon feedstock for biological fermenters used to culture microorganisms (e.g. bacterial, fungal, heterotrophic algae) both native and bio-engineered (GMO) for the ultimate production of a wide range of chemical and biochemical products, such as enzymes, functional proteins, carbohydrates, biopolymers, pharmacological compounds, pigments, oils and lipids, alcohols other than ethanol, polyols, isoprene, flavourings, fragrances, and long chain hydrocarbons.

### Reactor Configuration

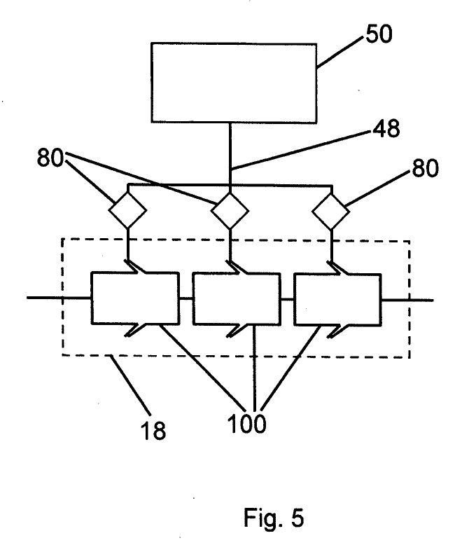

FIG. 4 depicts various configurations of the reactor 18 in FIG. 1, FIG. 8, FIG. 10, or FIG. 11. For clarity, the pipework necessary to connect a or each fluid mover to a source of transport fluid is omitted from the diagrams. In FIG. 4(a), reactor 18 consists of a single fluid mover 100. In FIG. 4(b), reactor 18 consists of three fluid movers 100 in series. FIG. 4(c) shows two fluid movers 100 in parallel and FIG. 4(d) shows two parallel legs, each consisting of two fluid movers 100 in series. These configurations are examples only, other numbers such as, e.g., from 1-100, including 1-50, such as 1-25 or 1-10, of fluid movers 100 in series or in parallel are possible, as required for the application of choice. Additional valves and pumps (not shown) may be included in order to control the flow as desired. For example, in order to apportion the slurry evenly where a number of fluid activation devices are in parallel, or so that one leg at a time of a parallel system can be closed off in order to allow cleaning in place (CIP). FIG. 5 shows the configuration depicted in FIG. 4(b) in more detail and incorporates the transport fluid supply 50 and the transport fluid supply line 48 that connects the transport fluid supply 50 to the three fluid movers 100. Incorporated in each transport fluid supply line 48 prior to each individual fluid mover 100 is a transport fluid conditioner 80. The transport fluid conditioner 80 may be adapted to vary the supply pressure of the transport fluid to each nozzle. Alternative transport fluid conditioners may be, e.g., a heating device to create superheated steam or a condensation trap to remove condensate from the transport fluid supply line 48. Similar pipework and transport fluid conditioners may be incorporated for any reactor 18 consisting of any configuration of fluid movers in parallel and/or in series. Additionally, one or more transport fluid supplies 50 may be utilised.

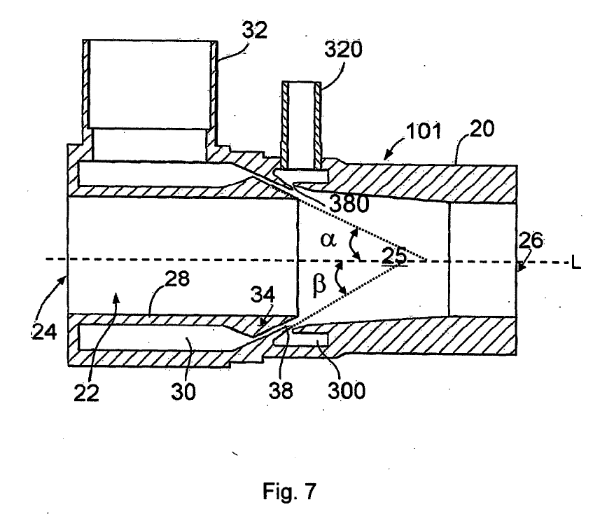

An alternative embodiment of a device according to the present invention that may act as a fluid mover is shown in detail in FIG. 7. The fluid mover 101 is substantially the same as the fluid mover 100 shown in FIG. 2, so like numbers have been used for like parts. The main difference is that the fluid mover 101 has an additional transport fluid inlet 320, transport fluid plenum 300 and transport fluid nozzle 380. The transport fluid nozzle 380 is a convergent-divergent nozzle similar to the transport fluid nozzle 38 described in FIG. 1 and operates in the same manner. In this embodiment, the transport fluid nozzles 38 and 380 are shown directly adjacent to each other, but they may be spaced apart along the length of the mixing region 25 in any manner. The angle β defines the angle of inclination of the leading edge of the divergent portion of the transport fluid nozzle 380 relative to the longitudinal axis L of the passage 22 as shown in FIG. 7. The angle α and the angle β are different in this embodiment, with angle α more acute than angle β. This relationship is not fixed, and one or other angle could be more acute, or they could be the same, depending on the requirements of the application. The angle β is preferably between 0° and 70°, more preferably between 0° and 30°. The embodiment shown in FIG. 7 has one additional transport fluid nozzle 380, however this is not limiting and more than one additional transport fluid nozzle may be included along the length of the mixing chamber. Transport fluid nozzles may be arranged in any configuration appropriate to accomplishing the desired task, e.g., liquefaction of starch-based biomass. For example, all transport fluid nozzles may be immediately adjacent to each other, or spaced along the length of the mixing chamber, or other arrangements (e.g. a series of pairs) as would occur to one skilled in the art. As required, each transport fluid nozzle may have its own transport fluid supply and transport fluid plenum, or some or all of the transport fluid nozzles may share these features.

Whilst the present invention need only utilise one fluid mover in the reactor, if the required process flow rate demands it the reactor may comprise a combination of fluid movers in series and/or parallel. This may also be the case with the temperature conditioning unit made up of one or more of such fluid movers.

The apparatus may also include one or more recirculation pipes which can selectively recirculate slurry from downstream of the fluid mover to upstream of the device, so that the slurry can pass through the device more than once, if necessary. Where included, the first vessel may also include such an arrangement so that slurry can pass through the first vessel more than once, if necessary. FIG. 6 shows part of the fluid processing apparatus 1 or 1000 of FIG. 1 or FIG. 8, respectively, with representative recirculation loops shown as dash-dot lines. For clarity, several of the features relevant to the vessel 2 as shown in FIG. 1 or FIG. 8 are omitted. In FIG. 6(a), there are two recirculation loops, either or both of which may be incorporated in the fluid processing apparatus. The first recirculation loop 68 recirculates the slurry through the vessel 2 using a pump 69, the valve 12 prevents the slurry from leaving the vessel until the appropriate conditions have been reached (e.g. slurry temperature). The valve 12 may also function to apportion the slurry such that some passes through the recirculation loop whilst some proceeds into the first supply line 16. Such a recirculation loop may be in addition to the motor 8 and agitator 6 shown in FIG. 1 or FIG. 8 or instead of them. An additional port (not shown) in the recirculation loop may be used to add the enzyme into the slurry rather than adding the enzyme to the first vessel 2 or mixing it with the working fluid prior to adding the working fluid to the first vessel 2. The second recirculation loop 74 in FIG. 6(a) is driven by a pump 72. Valves 70 and 76 close the recirculation loop 74 off from the pipework 16 and TCU 52 (not shown) so that slurry can be passed through the reactor 18 for a desired time or until a desired condition (e.g. slurry temperature or viscosity) is reached. The valve 76 may also apportion the slurry such that some continues to the TCU 52 (not shown) whilst the rest recirculates through the recirculation loop 74. FIG. 6(b) shows an alternative recirculation loop 78 that returns the slurry to the first vessel 2 after it has passed through the reactor 18.

Instead of having heaters such as heated water jackets, the first and/or second vessel may alternatively comprise an insulation layer on the exterior surface thereof. The insulation layer keeps the temperature of the slurry inside the vessel in the desired ranges stated above. The working fluid may be pre-heated by an external heater (not shown) prior to being mixed with the feedstock. The temperature of the slurry is maintained at the desired temperature in vessel 2 by either using the heated water jacket 4 or the insulation layer.

In addition to the agitator 60 and motor 62 in the second vessel 56, the second vessel may comprise a large number of internal baffles such that slurry is directed in a convoluted continuous flow path that slowly takes it through the vessel.

The low shear centrifugal pump which moves the slurry from the first vessel into the reactor may be replaced with any other suitable pump, such as either a membrane pump or a peristaltic pump, for example.

Whilst the TCU described above comprises one or more fluid movers of the type shown in FIG. 2, they may be replaced by a heat exchanger. The heat exchanger may be a shell and tube heat exchanger with the slurry passing through a tube and heated water passing through the shell surrounding the tube. Alternatively, the TCU may be replaced by a direct steam injection ‘sparge heater’ or a jacketed liquefaction tank.

The preferred concentration of the liquefaction enzyme in the slurry during development in the first vessel assumes an average of 10%-15% feedstock moisture content and an average starch content of 70%-75% dry weight.

Whilst the enzyme is preferably introduced to the slurry upstream of the fluid mover, the enzyme may also be introduced in the device or else, downstream of the device following activation of the starch content.

Whilst the illustrated embodiment of the invention includes both first and second vessels for handling the slurry, it should be appreciated that the invention need not include the vessels to provide the advantages highlighted above. Instead of a first vessel, the first hydrator may be a pipe or an in-line mixing device into which the feedstock, working fluid and enzyme are introduced upstream of the fluid mover. Similarly, the second vessel may be replaced by pipework in which the conversion of the activated starch to sugar takes place.

### Comparative Example

The example below describes the operation and performance of a typical plant (of the Dry Mill type) and compares this to the performance of the same plant after adding the apparatus and undertaking the process of the present invention.

Maize is supplied to the plant as grain and then ground to a flour. A conveyor feeds the flour to the slurry tank, where it is mixed with working fluid and continuously agitated (stirred). The plant working fluid is a combination of backset (approx. 25%) and process condensate (approx. 75%). The process condensate is heated before it enters the slurry tank in order to maintain the temperature of the slurry at 85° C.-88° C. Aqueous ammonia is added to the slurry tank in order to maintain a pH of approximately 6.0. This temperature range and pH are the preferred conditions for the enzyme α-amylase, which is also added to the slurry tank.

The slurry is pumped from the slurry tank, passed through a strainer to remove large particles (which are returned to the slurry tank) and then split into two streams, the first is returned to the slurry tank via a recirculation loop, the second stream continues to the liquefaction tank. The temperature and pH in the liquefaction tank are the same as those in the slurry. The liquefaction tank is divided into compartments with baffles so that the slurry passes slowly through the tank over a period of 90-120 minutes.

The mash, which consists of a liquid portion and a wet corn portion then leaves the liquefaction tank, a mash dilute may be added to maintain a consistent density. The mash is then cooled to about 32° C. in a mash cooler and then pumped to a fermenter. When the fermenter is about 5%-15% full of mash, a yeast prop is added. This is a pre-prepared mixture of 35% water and 65% mash to which is added yeast, gluco-amylase, urea (nitrogen to feed the yeast), zinc sulphate (speeds fermentation) and magnesium sulphate (aids yeast health), the proportions of each depend on the needs of the yeast. The yeast prop is held in a yeast mix tank with air bubbling through it for 10 hours prior to being added to the fermenter. During this time, a strong yeast mix forms containing a large number of colony forming units (approx. 500-600 million colony forming units per millilitre).

The plant uses a fermentation process known as Simultaneous Saccharification and Fermentation (SSF) whereby gluco-amylase is added to perform the saccharification step (breaking the dextrin and other short polysaccharide chains down to smaller sugar units such as glucose) the yeast then consumes the glucose to make ethanol. Too high a level of glucose stresses the yeast, and too low a level starves it, so gluco-amylase is added gradually (at the rates given in Table 2) throughout the fermenter fill time in order to maintain a constant glucose level in the mash. The total fermentation time (including 12 hours of fill time) is about 45-55 hours, after which the fermentation tank is drained and further treatment processes such as distillation occur.

The above plant was modified so that the apparatus of the present invention was installed after the slurry tank recirculation loop and before the liquefaction tank. The reactor 18 consisted of two parallel legs, each of which contained five in-series fluid movers of the type shown in FIG. 2, of which the last was operating as a TCU. Each leg fed separately into the liquefaction tank. Steam was injected into the slurry as it passed through the reactor 18 at a rate of 88.6 kg/min (195 lb./min.) at a maximum steam pressure of 6.5 bar gauge (94 psig) 7.5 bar absolute. The temperature of the slurry entering the reactor 18 was 48° C. and on entering the liquefaction tank was 84° C. At the end of the liquefaction process, the temperature of the mash was 83° C. and the DE value was 13.4 (compared to a typical value for this plant without the process of the present invention of 12.7). The process of the present invention achieved a higher DE value than the typical process with a lower level of dosing with α-amylase.

TABLE 1
Conditions in the slurry tank

| | Process of | Typical |
|---|---|---|
| | present invention | plant |
| Slurry flow rate (l/min) | 1620 | |
| | (430 gallon/min) | |
| Wet corn (% of slurry weight)* | 35.7% | 36.6% |
| α-amylase dosing rate (ml/min) | 140 | 175 |
| α-amylase dosing level (ml/kg corn) | 0.21 | 0.25 |
| Temperature (° C.) | 48 | 84 |

*Corn contains a certain amount of water (typically about 15%). The reason for the lower level of wet corn in the process of the present invention is that because it is able to activate more of the available starch, less corn is required for a given ethanol yield.

The conditions in the fermenter are given in Table 2. The gluco-amylase dosing level is initially higher for the process of the present invention (though both processes use the same total amount of the enzyme). This is because the altered proportions of solids and liquids and the balance of sugars in the mash suited the yeast, such that the rates of yeast growth and ethanol production at the start of the fermentation process were accelerated. The yeast, therefore, required a faster rate of glucose release to feed it, so the initial dosing levels of gluco-amylase compared to the typical process were increased.

TABLE 2
Conditions in the fermentation tank

| | Process of | Typical |
|---|---|---|
| | present invention | plant |
| Fermentation tank fill volume | 1476000 | |
| (litre) | (390,000 gallon) | |
| Gluco-amylase dosing rate | 600 ml/min for 6 | 480 ml/min for 12 |
| | hours then | hours |
| | 360 ml/min for 6 | |
| | hours | |
| Ethanol produced | 0.308 | 0.295 |
| (kg ethanol/kg wet corn) | | |
| Ethanol produced | 2.61 | 2.51 |
| (gallons ethanol/bushel corn) | | |

A typical ethanol plant producing 40 million gallons of ethanol per year has to purchase 15.94 million bushels of corn. Table 2 shows that the process of the present invention gives a higher ethanol yield per bushel of corn, so less corn is required (15.33 million bushels) to produce the same amount of ethanol. At a purchase price of $4 per bushel of corn, this is a saving of $2.44 million per year. The process of the present invention also required less α-amylase for the liquefaction stage, providing a further cost saving. Energy savings due to the reduced heat requirements of the slurry tank are also possible.

Further improvements and modifications may be incorporated without departing from the scope of the present invention.

What is claimed is:

1. A process for the treatment of a starch-based feedstock, comprising: (a) mixing together a starch-based feedstock and a working fluid to form a slurry; (b) hydrating the starch-based feedstock with the working fluid; (c) adding an enzyme to the slurry; (d) heating and maintaining the slurry in a vessel at a temperature in the range of 55° C.-85° C. for a predetermined period of time (e) directing the slurry into a substantially constant diameter passage of a fluid mover; and (f) injecting a high velocity transport fluid into the slurry through one or more nozzles communicating with the passage, thereby further hydrating and heating the starch-based feedstock and dispersing the starch content of the slurry, whereby at least a portion of the slurry is atomized to form a dispersed droplet flow regime downstream of the one or more nozzles.

2. The process of claim 1, wherein step (f) comprises: (a) forming a low pressure region downstream of the one or more nozzles; and (b) generating a condensation shock wave within the passage downstream of the one or more nozzles by condensation of the transport fluid.

3. The process of claim 1, wherein the transport fluid is a hot, compressible gas.

4. The process of claim 3, wherein the hot, compressible gas is selected from the group consisting of steam, carbon dioxide, and nitrogen.

5. The process of claim 1, wherein the working fluid is water.

6. The process of claim 1, wherein the feedstock is selected from the group consisting of dry milled maize, dry milled wheat, dry milled sorghum, potato, oats, barley, rye, rice and cassava.

7. The process of claim 1, wherein the transport fluid is injected at a subsonic or a supersonic velocity.

8. The process of claim 1, wherein step (f) occurs on a single pass of the slurry through the fluid mover.

9. The process of claim 1, wherein step (f) includes recirculating the slurry through the fluid mover.

10. The process of claim 1, further comprising the step of recirculating the slurry through the vessel.

11. The process of claim 1, further comprising the step of passing the slurry through a strainer prior to step (e) to remove large particles and/or other debris from the slurry.

12. The process of claim 1, further comprising the step of passing the slurry through a jet cooker prior to step (e).

13. The process of claim 1, further comprising: (a) directing the slurry from the fluid mover to one or more residence tubes; (b) directing the slurry from the one or more residence tubes to a second fluid mover having a second substantially constant diameter passage; and (c) injecting a high velocity transport fluid into the slurry through one or more nozzles communicating with the passage of the second fluid mover, thereby further hydrating and heating the starch content of the slurry, whereby the slurry is further atomized to form a dispersed droplet flow regime downstream of the one or more nozzles of the second fluid mover.

14. The process of claim 13, further comprising the step of transferring the slurry to a second vessel from the second fluid mover, and maintaining the temperature of the slurry in the second vessel for another predetermined period of time.

15. The process of claim 1, the process further comprising the step of transferring the slurry to a second vessel from the fluid mover, and maintaining the temperature of the slurry in the second vessel for another predetermined period of time.

16. The process of claim 15, wherein the step of transferring the slurry to the second vessel includes passing the slurry through a low pressure flash tank to reduce the temperature of the slurry.

17. The process of claim 15, further comprising the step of agitating the slurry in the first and second vessels for the respective predetermined periods of time.

18. The process of claim 15, further comprising the steps of directing mash resulting from liquefaction of the slurry in the second vessel to a fermenter.

19. The process of claim 18, further comprising distilling ethanol after fermentation of the mash.

20. The process of claim 19, further comprising processing stillage resulting from the fermentation and distillation to produce backset that is directed to the first vessel.

21. The process of claim 15, further comprising the step of adding an enzyme composition to the slurry in the second vessel.

22. The process of claim 21, wherein the enzyme composition comprises α-amylase.

23. The process of claim 21, further comprising the step of adding a further enzyme to the slurry in the second vessel to break the starch content into short chains of polysaccharides in order to produce non-ethanol products.

24. An apparatus for treating a starch-based feedstock, the apparatus comprising: (a) a hydrator/mixer for (i) mixing and hydrating the feedstock with a working fluid to form a slurry, and (ii) maintaining the slurry at a temperature in the range of 55° C.-85° C. for a predetermined period of time; and (b) a fluid mover in fluid communication with the hydrator/mixer; wherein the fluid mover comprises: (i) a passage of substantially constant diameter having an inlet in fluid communication with the hydrator/mixer and an outlet; and (ii) a transport fluid nozzle communicating with the passage and adapted to inject high velocity transport fluid into the passage, whereby at least a portion of the slurry is atomized to form a dispersed droplet flow regime downstream of the nozzle.

25. The apparatus of claim 24, wherein the hydrator/mixer comprises a heated water jacket surrounding a first vessel having an outlet in fluid communication with the inlet of the passage.

26. The apparatus of claim 25, further comprising a second vessel having an inlet in fluid communication with the outlet of the passage.

27. The apparatus of claim 26, wherein the second vessel includes an insulator for insulating the contents of the second vessel.

28. The apparatus of claim 24, further comprising a residence tube section having an inlet in fluid communication with the outlet of the passage.

29. The apparatus of claim 28, wherein the residence tube includes an insulator for insulating the contents of the residence tube as it passes through.

30. The apparatus of claim 24, wherein the transport fluid nozzle is annular and circumscribes the passage.

31. The apparatus of claim 24, wherein the transport fluid nozzle has an inlet, an outlet and a throat portion intermediate the inlet and the outlet, wherein the throat portion has a cross sectional area which is less than that of the inlet and the outlet.

32. The apparatus of claim 24, further comprising a transport fluid supply adapted to supply transport fluid to the transport fluid nozzle.

33. The apparatus of claim 32, further comprising a plurality of fluid movers in series and/or parallel with one another, wherein the transport fluid supply is adapted to supply transport fluid to the transport fluid nozzle of each device.

34. The apparatus of claim 33, further comprising a plurality of transport fluid supply lines connecting the transport fluid supply with each nozzle, wherein each transport fluid supply line includes a transport fluid conditioner.

35. The apparatus of claim 34, wherein the transport fluid conditioner is adapted to vary the supply pressure of the transport fluid to its respective nozzle.

36. The apparatus of claim 33, further comprising a dedicated transport fluid supply for each transport fluid nozzle.

37. The apparatus of claim 36, wherein each transport fluid supply includes a transport fluid conditioner.

38. The apparatus of claim 37, wherein each conditioner is adapted to vary the supply pressure of the transport fluid to each respective nozzle.

39. The apparatus of claim 24 further comprising a low pressure flash tank located downstream of the fluid mover, the low pressure flash tank adapted to reduce the temperature of fluid leaving the passage of the fluid mover.

40. The apparatus of claim 24 further comprising a recirculation pipe adapted to allow fluid recirculation from downstream of the fluid mover to upstream of the fluid mover.

41. The apparatus of claim 24 further comprising a recirculation pipe adapted to allow fluid recirculation through the hydrator/mixer.

42. The apparatus of claim 24 further comprising a strainer located upstream of the fluid mover, the strainer adapted to remove large particles and/or other debris from the slurry.

43. The apparatus of claim 42, further comprising a jet cooker located downstream of the strainer and upstream of the fluid mover.

44. The apparatus of claim 43 further comprising: (a) one or more residence tubes located downstream of the fluid mover; (b) a second fluid mover located downstream of the one or more residence tubes, the second fluid mover comprising: (i) a passage of substantially constant diameter; and (ii) a transport fluid nozzle communicating with the passage of the second fluid mover and adapted to inject high velocity transport fluid into the passage of the second fluid mover, whereby the slurry is further atomized to form a dispersed droplet flow regime downstream of the nozzle of the second fluid mover.

45. The apparatus of claim 24, which is integrated into an ethanol production plant for producing ethanol from the starch-based feedstock.

46. The apparatus of claim 24, which is integrated into an ethanol production plant for producing non-ethanol products from the starch-based feedstock.

47. A system for producing ethanol comprising an apparatus according to claim 24 integrated into an ethanol production plant,

48. The system of claim 47, further comprising a fermenter, a yeast prop tank coupled to the fermenter, a beer well located downstream of the fermenter, a beer column, a centrifuge adapted to process stillage from the beer column, and a thin stillage tank located downstream of the centrifuge and adapted to produce backset to be directed to the hydrator/mixer.

49. The system of claim 47, wherein the ethanol production plant is a dry mill or a wet mill plant.

50. The system of claim 49, wherein the dry mill plant utilizes a corn dry grind based feedstock.

51. The system of claim 49, wherein the wet mill plant utilizes a corn wet milling based feedstock.

52. A process for calculating ethanol yield during the production of biofuels in a plant, the process comprising: (a) establishing a composition of dry matter and water making up a mass unit of mash which is hydrolysed prior to entering into a fermenter that is part of an ethanol production system within the plant; (b) using a computer programmed to process inputs from the production system to calculate a mass of dry matter and a mass of wet matter making up the mass unit (c) calculate an amount of wet corn in the mass unit by adding the mass of dry matter and the mass of wet matter; (d) using the computer to calculate an amount of ethanol produced from the mass unit based on stoichiometry and measurements of materials in the production system; and (e) determine the ethanol yield by dividing the calculated amount of ethanol by the calculated amount of wet corn.

53. The process of claim 52, wherein the measurements of materials comprise measurements of materials entering into and leaving the fermenter.

54. The process of claim 52, wherein the measurements of materials comprise relied ethanol concentration, dissolved solids concentration, water mass balances and beer density.

55. A process for improving ethanol yield during the production of biofuels in a plant, the process comprising: (a) calculating the ethanol yield using the process of claim 52; (b) adjusting one or more parameters to improve yield.

56. The process for improving ethanol yield of claim 55, wherein the adjusting one or more parameters comprises adjusting at least one input.

57. The process for improving ethanol yield of claim 56, wherein the at least one input is selected from the group consisting of an amount of feedstock, an amount of liquid and an amount of enzyme.

58. The process for improving ethanol yield of claim 55, wherein the adjusting one or more parameters comprises adjusting at least one operating condition.

59. The process for improving ethanol yield of claim 58, wherein the at least one operating condition is selected from the group consisting of temperature, process time, process flow rate, throughput, fluid speed and pH level.
